(19) United States
(12) Patent Application Publication
(10) Pub. No.: US 2002/0098179 A1
(43) Pub. Date: Jul. 25, 2002

(54) Pharmaceutical combinations

(76) Inventors: Christopher John Brearley, Sandwich (GB); Paul Butler, Sandwich (GB); Suresh Babubhai Chahwala, Sandwich (GB); Michael Chopp, Sandwich (GB); Michael Krams, Sandwich (GB); Michael Looby, Sandwich (GB); Fiona MacIntyre, Sandwich (GB); Andrew Brian McElroy, Sandwich (GB); Aileen Dorothy McHarg, Sandwich (GB)

Correspondence Address: Paul H. Ginsburg, Pfizer Inc., 20th Floor, 235 East 42nd Street, New York, NY, 10017-5755, US

(21) Appl. No.: 09/969,271

(22) Filed: Oct. 1, 2001

Related U.S. Application Data
(60) Provisional application No. 60/253,847, filed on Nov. 29, 2000.

(30) Foreign Application Priority Data
Oct. 17, 2000 (GB) 0025473.0

(51) Int. Cl. A61K 38/48; A61K 38/17

(52) U.S. Cl. 424/94.64; 514/12

(57) ABSTRACT

This invention relates, inter alia, to methods of treating pathophysiological conditions involving neutrophils, comprising administering to a patient in need of such treatment a combination therapy comprising at least one Neutrophil Inhibitory Factor (NIF) and at least one other agent that protects neurons from toxic insult, inhibits the inflammatory reaction after brain damage or promotes cerebral reperfusion (i.e. neuroprotective or thrombolytic/fibrinolytic agents), or a pharmaceutically acceptable salt thereof.

### TECHNICAL FIELD

The present invention relates, inter alia, to methods of treating pathophysiological conditions involving neutrophils, comprising administering to a patient in need of such treatment a combination therapy comprising at least one Neutrophil Inhibitory Factor (NIF) and at least one other agent that protects neurons from toxic insult, inhibits the inflammatory reaction after brain damage or promotes cerebral reperfusion (i.e. neuroprotective or thrombolytic/fibrinolytic agents), or a pharmaceutically acceptable salt thereof.

### BACKGROUND OF THE INVENTION

Leukocytes are a class of cells comprised of lymphocytes, monocytes and granulocytes. The lymphocytes include within their class, T-cells (as helper T-cells and cytotoxic or suppressor T-cells), B-cells (as circulating B-cells and plasma cells), natural killer (NK) cells and antigen-presenting cells. Monocytes include within their class, circulating blood monocytes, Kupffer cells, intraglomerular mesangial cells, alveolar macrophages, serosal macrophages, microglia, spleen sinus macrophages and lymph node sinus macrophages. Granulocytes include within their class, neutrophils, eosinophils, basophils, and mast cells (as mucosa-associated mast cells and connective tissue mast cells). Thus, neutrophils and eosinophils are a subset of leukocytes.

Neutrophils are an essential component of the host defence system against microbial invasion. In response to soluble inflammatory mediators released by cells at the site of injury, neutrophils migrate into tissue from the bloodstream by crossing the blood vessel wall. At the site of injury, activated neutrophils kill foreign cells by phagocytosis and/or by the release of cytotoxic compounds, such as oxidants, proteases and cytokines. Despite their importance in fighting infection, neutrophils themselves can promote tissue damage. During an abnormal inflammatory response, neutrophils can cause significant tissue damage by releasing toxic substances at the vascular wall or in uninjured tissue. Alternatively, neutrophils that adhere to the capillary wall or clump together in venules may produce tissue damage by ischaemia (“no reflow” phenomenon). Such abnormal inflammatory responses have been implicated in the pathogenesis of a variety of clinical disorders including adult respiratory distress syndrome (ARDS); ischaemia-reperfusion injury following myocardial infarction, shock, stroke, and organ transplantation; acute and chronic allograft rejection; vasculitis; sepsis; rheumatoid arthritis; and inflammatory skin diseases (Harlan et al., 1990 Immunol. Rev. 114, 5).

Stroke is the most common neurologic disorder and ranks third, after cancer and heart disease, as the cause of death in Western Europe and North America. The incidence of stroke rises sharply with age in both men and women (for each decade after the age of 55, the risk of stroke doubles), with most strokes occurring in the 65-75 age group. Moreover, stroke has a disproportionate effect on women who account for approximately 43% of the strokes that occur each year, yet account for 62% of stroke deaths (National Stroke Association, Brain Attack Statistics (http;//www.stroke.org)). In the USA alone, it is estimated that there are 730,000 new or recurrent cases of stroke each year resulting in approximately 160,000 deaths per annum. The USA has now over four million survivors coping with its debilitating consequences (National Stroke Association, Brain Attack Statistics (http;//www.stroke.org)).

The size and location of the infarct to a large extent determines the clinical manifestations of ischaemic stroke and an evolving neurological deficit is one of the key diagnostic features of the disease. These symptoms range from blurred vision to vertigo, dizziness, convulsions and loss of consciousness, which are associated with a wide range of motor and sensorimotor deficits, including tremor, lack of motor co-ordination and partial paralysis. In addition, higher cortical dysfunction may occur which is manifested as amnesia, dementia and delirium, as well as language and speech disturbances. It is possible that patients surviving a stroke may be severely mentally and physically disabled (National Stroke Association, Brain Attack Statistics (http;//www.stroke.org); National Institute of Neurological Disorders and Stroke. Stroke (Brain Attack) (http://www.hinds.nih.gov) (1997)).

Stroke, or focal ischaemic brain injury, is an outward manifestation of a localised, sudden interruption of the blood supply to some part of the brain system (but most often in the territory of the middle cerebral artery). The most common types of stroke are the formation of a clot in a cerebral vessel (cerebral infarction, which affects 80% of patients), rupture of a blood vessel in the brain (primary intracerebral haemorrhage which affects 15% of patients) and rupture of a blood vessel around the brain (subarachnoid haemorrhage, which affect 5% of patients) (Vaughn J and Bullock R (1999) Cellular and Vascular Pathophysiology of Stroke. In: L. Miller (Ed) Stroke Therapy, Basic, Preclinical and Clinical Directions). If blood flow is not restored within a short period of time after a stroke, this will lead to a core of severely ischaemic brain tissue that may not be salvaged. However, the ultimate size of the brain infarct also depends on the penumbra, a zone of tissue around the core of the infarct where neuronal electrophysiology is not compromised and blood flow is still maintained above a critical level (Fisher M. Stroke 1997;28:866-872). If blood flow in this penumbral zone further decreases and/or energy requirements are exceeded, the infarct zone will inevitably expand. There is now good evidence that the penumbra exists in human stroke patients (Read S J et al Neurology 1998;51:1617-1621), but the extent and temporal dynamics of this area are less well defined (Kaufmann A M et al., Stroke 1999;30:93-99). However, a recent clinical study suggests that the penumbra as well as sufficiently perfused brain tissue can account for up to 30% of the final infarct volume in stroke patients (Heiss W D et al Stroke 1999;30:1486-1489). It is, thus, evident that the prime goal of neuroprotection is to salvage the ischaemic penumbra.

Considerable research effort has been devoted to the identification of the effectors and the sequence of events that lead to neuronal cell death following cerebral ischaemia. Among the initial events are the widespread neuronal depolarisation and massive release of glutamate, which activates N-methyl-D-aspartate (NMDA) receptors, leading to calcium influx (Lee J M et al., Nature 1999;399:A7-A14). Numerous secondary processes occur thereafter to amplify the ischaemic neuronal damage, leading to activation of proteases, phospholipases, nitric oxide synthase, protein kinases and the generation of highly active free radicals which can lead to an inflammatory cell response via the release of cytokines (Lee J M et al., Nature 1999;399:A7-A14; Dirnagl U et al TINS 1999;22:391-397; Barone F C and Feuerstein G Z. J Cereb Blood Flow Met. 1999;19:819-834). The result of this ischaemic cascade is that brain cells that have undergone cellular injury can die by either necrosis or apoptosis (programmed cell death), a form of cell suicide (Liu P K et al (1999) Apoptosis: DNA Damage and Repair in Stroke. In: L. Miller (Ed) Stroke Therapy, basic, preclinical and clinical directions), depending on the nature and intensity of the stimulus and the type of cell at risk (Leist M and Nicotera P. Exp Cell Res 1998;239:183-201). It is now thought that necrosis is the predominant mechanism of cell death in the ischaemic core, whereas in the penumbra, where milder injury occurs (see above), cell suicide becomes unmasked and neuronal death resembles apoptosis (Lee J M et al., Nature 1999), which may be blocked by anti-apoptotic compounds (Schultz J B et al Ann Neurol 1999;45:421 -429).

Acute stroke treatment involves two major approaches. Firstly, therapy designed to restore or improve cerebral blood flow by dissolving the embolus or thrombus that caused the artery occlusion (thrombolysis). Secondly, therapy focused on the biochemical and metabolic consequences of ischaemic brain injury in order to prevent neuronal cell death in the penumbra (neuroprotection).

The first approach targets the shortfall of available arterial oxygen and glucose relative to the needs of local brain tissue by enhancing blood flow by the lyses of an arterial thrombus (Zivan J A Neurology 1999;53:14-19). Early thrombolysis, using intravenous recombinant tissue plasminogen activator (t-PA), is currently the only approved therapy for stroke (The National Institute of Neurological Disorders and Stroke rT-PA Stroke Study Group. New Engl J Med 1995;333:1581-1587). The thrombolytic needs to be given within 3.0 hrs of the onset of symptoms and the application of such therapy is severely constrained by the necessity to utilise expensive computerised tomographic (CT) scanning to exclude the possibility of haemorrhagic stroke, for which such agents are contraindicated because they would exacerbate bleeding (Clarke W. AHA Stroke Conference, Nashville Tenn. 1999). With such constraints, it is estimated that around 5% of 500,000 stroke patients currently receive thrombolytic therapy (Clarke W. AHA Stroke Conference, Nashville Tenn. 1999). More recently, two other agents were shown to have therapeutic efficacy: the thrombolytic agent pro-urokinase (r-Pro-UK), delivered by intra-arterial catheter directly to an intravascular thrombus, and the proteolytic enzyme ancrod which has fibrinogen lowering properties (Goldberg MP Stroke Trial database, Internet Stroke Centre at Washington University (http//www.neuro.wustl.edu/stroke) (1997)). However, both these agents are likely to have the same restricted use as t-PA.

The second therapeutic approach, neuroprotection, aims to reduce the intrinsic vulnerability of brain tissue to ischaemia, a strategy that might be used in both ischaemic and haemorrhagic strokes (as the latter invariably involves an ischaemic component). Thus, neuroprotective approaches have focused mainly on blocking excitotoxicity, that is, neuronal cell death triggered by the excitatory transmitter glutamate, and mediated by cytotoxic levels of calcium influx (Fisher M 1999). There are at present more than 30 different clinical trials at various phases that involve potential neuroprotective compounds, and most of these either directly (for example, glutamate receptor antagonist) or indirectly (for example, blockers of voltage-gated sodium or calcium channels) attenuate excitotoxicity (Devuyst G and Bogousslavsky J Curr Opin Neurol 1999;12:73-79). However, negative results from several recent trials with antagonists of NMDA type glutamate receptors (Drug and Market Development 1 March 1999; The Genesis Report February 1998) challenges the hypothesis that excitotoxicity with attendant neuronal calcium overload is the predominant mechanism underlying ischaemic neural injury. A possible key reason for these failures is that adequate blood levels of the agent could not be achieved due to adverse side-effects (Lee J M et al., Nature 1999). Another possible explanation for the difficulties in demonstrating therapeutic benefits with these compounds is that changes associated with these targets occur very soon after stroke. Calcium changes happen within seconds to minutes after the ischaemic insult. Glutamate release has been demonstrated to maximum after 20-40 minutes, and most of the damage associated with its excitotoxicity may occur within the first 60 minutes (Dirnagl U et al TINS 1999;22:391-397). Thus, by the time the patient is available for treatment in the emergency room these mechanisms of damage might be largely exhausted. Indeed, data from a recent clinical study suggested that the average time for enrolment of stroke patients into the study was 12 hours after the onset of symptoms (DeGraba T J et al., Stroke 1999;30:1208-1212). Therefore, if neuroprotective strategies are to be efficacious in humans, there needs to be a broadening of therapeutic targeting beyond excitotoxicity and neuronal calcium overload to develop neuroprotective strategies with a wider therapeutic window for effective intervention without causing adverse side-effects.

Delayed cell death following cerebral ischaemia has an apoptotic component for which many biochemical pathways amenable to pharmacological intervention have been identified (Kinloch et al TIPS 1999;20:25-42). One such pathway is the stress-activated protein kinases (SAPK e.g. p38 and JNK). SAPK play an important role in transducing stress-related signals by a cascade mechanism of phosphorylation of intracellular kinases and transcription factors (Robinson and Cobb Curr Opin Cell Biol 1997;9:180-186) that regulate cell survival, apoptosis and inflammatory cytokine production (Tibbles L A and Woodgett J R Cell Mol Life Sci 1999;55:1230-1254). Recently, evidence has emerged to suggest that the JNK pathway is indeed important for neuronal apoptosis (Yang D D et al., Nature 1997;389:865-870). These studies place activation of the JNK pathway at an early stage, acting to increase Fas ligand expression and subsequent Fas receptor-mediated cell death (Fas is a member of the family of cell death receptors which are part of the tumour necrosis factor superfamily) (Herdegen T et al J Neurosci 1998;18(14):5124-5135). In addition, it is now apparent that activation of the JNK pathway, can be mediated via phosphorylation of JNK (JNK1, 2 or 3) by either MKK7 or MKK4 or both. Recent reports suggest that MKK7 rather than MKK4 may be an appropriate target for neuronal apoptosis.

Neutrophil adhesion at the site of inflammation is believed to involve at least two discrete cell-cell interactive events. Initially, vascular endothelium adjacent to inflamed tissue becomes adhesive to neutrophils; neutrophils interact with the endothelium via low affinity adhesive mechanisms in a process known as “rolling”. In the second adhesive step, rolling neutrophils bind more tightly to vascular endothelial cells and migrate from the blood vessel into the tissue.

Neutrophil rolling along affected vascular segments and other initial low affinity contacts between neutrophils and the endothelium are reported to be mediated by a group of monomeric, integral membrane glycoproteins termed selecting. All three of the selectins so far identified, that is L-selectin (LECAM-1 or LAM-1) present on the surface of neutrophils, E-selectin (endothelial leukocyte adhesion molecule-1 or ELAM-1) present on endothelial cells and P-selectin (granule membrane protein-140, GMP-140, platelet activation-dependent granule-external membrane protein, PADGEM or CD62) expressed on endothelial cells, have been implicated in neutrophil adhesion to the vascular endothelium (Jutila et al., 1989 J. Immunol 143, 3318; Watson et al., 1991 Nature 349, 164; Mulligan et al., 1991 J. Clin. Invest. 88, 1396; Gundel et al., 1991 J. Clin. Invest. 88, 1407; Geng et al., 1990 Nature 343, 757; Patel et al., 1991 J. Cell Biol. 112, 749). The counter-receptor for E-selectin is reported to be the sialylated Lewis X antigen (sialyl-Lewis^{x}) that is present on cell-surface glycoproteins (Phillips et al., 1990 Science 250, 1130; Walz et al., 1990 Science 250, 1132; Tiemeyer et al., 1991 Proc. Natl. Acad. Sci.(USA) 88, 1138; Lowe et al., 1990 Cell 63, 475). Receptors for the other selectins are also thought to be carbohydrate in nature but remain to be elucidated.

The more stable secondary contacts between neutrophils and endothelial cells are reported to be mediated by a class of cell adhesion molecules known as integrins. Integrins comprise a broad range of evolutionarily conserved heterodimeric transmembrane glycoprotein complexes that are present on virtually all cell types. Members of the leukocyte-specific CD18 (β₂) family of integrins, which include CD11a/CD18 (LFA-1) and CD11b/CD18 (Mac-1, Mo-1 or CR3) have been reported to mediate neutrophil adhesion to the endothelium (see Larson and Springer, 1990 Immunol Rev. 114, 181;Gahmberg et al Eur. J. Biochem. 1997;245:215-232).

Endothelial cell counter-receptors for these integrins are the intercellular cell adhesion molecules ICAM-1 and ICAM-2 for CD11a/CD18 and ICAM-1 for CD11b/CD18, respectively (Rothlein et al., 1986 J. Immunol. 137, 1270; Staunton et al., 1988 Cell 52, 925; Staunton et al., 1989 Nature 339, 61). The ICAMs are monomeric transmembrane proteins that are members of the immunoglobulin superfamily.

The CD11b/CD18 integrin is expressed on a variety of leukocytes, including monocytes, macrophages, granulocytes, large granular lymphocytes (NK cells), and immature and CD5⁺ B cells (Kishimoto, T. K., Larson, R. S., Corbi, A. L., Dustin, M. L., Staunton, W E., and Spriger, T. A. (1989) Adv. in Immunol. 46,149-182).

CD11b/CD18 has been implicated in a variety of leukocyte functions including adhesion of neutrophils to endothelial cells (Prieto, J., Beatty, P. G., Clark, E. A., and Patarroyo, M. (1988) Immunology 63, 631-637; Wallis, W. J., Hickstein, D. D., Schwartz, B. R., June, C. H., Ochs, H. D., Beatty, P. G., Klebanoff, S. J., and Harlan, J. M. (1986) Blood 67, 1007-1013; Smith, C. W., Marlin, S. D., Rothlein, R., Toman, C., and Anderson, D. C. (1989) J. Clin. Invest. 83, 2008-2017) and release of hydrogen peroxide from neutrophils (Shappell, S. B., Toman, C., Anderson, D. C., Taylor, A. A., Entman, M. L. and Smith, C. W. (1990) J. Immunol. 144, 2702-2711; Von Asmuth, E. J. U., Van Der Linden, C. J., Leeuwenberg, J. F. M., and Buurman, W. A. (1991) J. Immunol. 147,3869-3875). This integrin may play a roll in neutrophil and monocyte phagocytosis of opsonized (i.e. C3bi-coated) targets (Beller, D. I., Springer, T. A., and Schreiber, R. D. (1982) J.Exp. Med. 156,1000-1009). It has also been reported that CD11b/CD18 contributes to elevated natural killer activity against C3bi-coated target cells (Ramos, O. F., Kai, C., Yefenof, E., and Klein, E. (1988) J. Immunol. 140,1239-1243).

The activation of endothelial cells and neutrophils is believed to represent an important component of neutrophil-mediated inflammation (see Berton et al Int. J.Clin. Lab. Res. 1996;26:160-177). Factors that induce cell activation are termed agonists. Endothelial cell agonists, which are believed to include small regulatory proteins such as tumour necrosis factor (TNFα) and interleukin-Iα (IL-1α), are released by cells at the site of injury. Activation of endothelial cells has been reported to result in the increased surface expression of ICAM-1 (Staunton et al., 1988 Cell 52, 925) and ELAM-1 (Bevilacqua et al., 1987 Proc. Natl. Acad. Sci.(USA) 84, 9238). Raised levels of expression of these adhesive molecules on the surface of activated endothelial cells is believed to lead to the observed increased adhesivity of neutrophils for the vascular endothelium near sites of injury.

Activation of the neutrophil results in profound changes to its physiological state, including shape change, ability to phagocytose foreign bodies and release of cytotoxic substances from intracellular granules. Moreover, activation is believed to greatly increase the affinity of adhesive contacts between neutrophils and the vascular endothelium, perhaps through a conformational change in the CD11b/CD18 integrin complex on the neutrophil surface (Vedder and Harlan, 1988 J. Clin. Invest. 81, 676; Buyon et al., 1988 J. Immunol. 140, 3156). Factors that have been reported to induce neutrophil activation include IL-1α, granulocyte/monocyte-colony stimulating factor (GM-CSF), granulocyte-colony stimulating factor (G-CSF), MIP-1, interleukin-8 (IL-8), TNFα, the complement fragment C5a, the microbe-derived peptide formyl-Met-Leu-Phe, the lipid-like molecules leukotriene B4 (LTB₄), and platelet activating factor (Fuortes and Nathan, 1992, in Molecular Basis of Oxidative Damage by Leukocytes, eds Jesaitis, A. J. and Dratz, E. A. (CRC Press) pp. 81-90). In addition, phorbol esters (e.g., phorbol 12-myristate 13-acetate; PMA) have been proposed as a potent class of synthetic lipid-like neutrophil agonists. With the exception of PMA, these agonists are believed to activate neutrophils by binding to receptors on their surface. Receptors that are occupied by agonist molecules are believed to initiate, within the neutrophil, a cascade of events that ultimately will result in the physiological changes that accompany neutrophil activation. This process is known as signal transduction. The lipid-like PMA is proposed to affect neutrophil activation by passing through the plasma membrane at the cell surface and directly interacting with intracellular components (i.e. protein kinase) of the signal transduction machinery.

There exist two general classes of compounds that have been reported to down-regulate the function of neutrophils, and these compounds have been shown to mitigate inflammation. One group of anti-inflammatory compounds has been proposed to function as inhibitors of neutrophil activation, and presumably adhesion, by acting on components of the signal transduction machinery. A second class of anti-inflammatory compounds has been proposed to block neutrophil infiltration into inflammatory foci by acting as direct inhibitors of the adhesive receptors that mediate contact between neutrophils and the vascular endothelium.

Many of the anti-inflammatory compounds currently used as therapeutics, including prostaglandins, catecholamines, and a group of agents known as non-steroidal anti-inflammatory drugs (NSAIDs), are believed to fall into the first category (Showell and Williams, 1989, in Immunopharmacology, eds. Gilman, S. C. and Rogers, T. J. [Telford Press, NJ] pp 23-63). For example, the enhanced adhesiveness observed for TNFα-activated neutrophils has been reported as associated with decreased levels of a mediator of signal transduction, cyclic AMP (cAMP) (See Nathan and Sanchez, 1990 JCB 111, 2171). Exposure of neutrophils to prostaglandins and catecholamines has been correlated with elevated levels of intracellular cAMP (Showell and Williams, 1989). While signal transduction inhibitors have been used extensively as anti-inflammatory therapeutic agents, they have been shown to have several disadvantages including poor efficacy in acute inflammatory conditions, lack of specificity and undesirable side-effects such as gastric or intestinal ulceration, disturbances in platelet and central nervous system function and changes in renal function (Insel, 1990 in The Pharmacological Basis of Therapeutics, eds. Gilman, A. G., Rail, T. W., Nies, A. S., and Taylor, P. [Pergamon, N.Y.], 8th Ed., pp. 638-681).

Glucocorticoids have long been recognised for their anti-inflammatory properties. Steroid induced inhibition of neutrophils has been reported for several neutrophil functions, including adhesion (Clark et al., 1979 Blood 53, 633-641; MacGregor, 1977 Ann. Intern. Med. 86, 35-39). The mechanisms by which glucocorticoids modulate neutrophil function are not well understood, but they are generally believed to involve the amplification or suppression of new proteins in treated neutrophils that play a key role in the inflammatory process (Knudsen et al., 1987 J. Immunol. 139, 4129). In particular, a group of proteins known as lipocortins, whose expression is induced in neutrophils by glucocorticoids, has been associated with anti-inflammatory properties (Flower, 1989 Br. J. Pharmacol. 94, 987-1015). Lipocortins may exert anti-neutrophil effects by interacting with sites on the neutrophil surface (Camussi et al., 1990 J. Exp. Med. 171, 913-927), but there is no evidence to suggest that the lipocortins act by directly blocking adhesive proteins on the neutrophil. Apart from their beneficial anti-inflammatory properties, glucocorticoids have been associated with significant side effects. These include suppression of pituitary-adrenal function, fluid and electrolyte disturbances, hypertension, hyperglycaemia, glycosuria, susceptibility to infection, ulcers, osteoporosis, myopathy, arrest of growth and behavioural disturbances (Insel, 1990).

A second class of anti-inflammatory compounds which are reported as direct inhibitors of neutrophil adhesion to the vascular endothelium are monoclonal antibodies.

Monoclonal antibodies that recognise and block ligand-binding functions of some of these adhesive molecules have been reported to act as in vivo inhibitors of neutrophil-mediated inflammation. In particular, monoclonal antibodies to the CD18 subunit of the CD18 integrin complexes (i.e., CD11a/CD18, CD11b/CD18,CD11c/CD18 and CD11d/CD18 (Plow et al. 2000 J. Biol. Chem. 275 (29), 21785-21788)) on the surface of neutrophils have been reported to prevent a variety of neutrophil-mediated tissue injury in animal models, including pulmonary oedema induced by reperfusion (Horgan et al, 1990 Am. J. Physiol. 259, L315-L319), organ injury induced by haemorrhagic shock (Mileski et al, 1990 Surgery 108, 206-212), myocardial damage following ischaemia/reperfusion (Winquist et al, 1990 Circulation 111-701), oedema and tissue damage following ischaemia/reperfusion of the ear (Vedder et al, 1990 Proc. Natl. Acad. Sci.(USA) 87, 2643-2646), brain oedema and death produced by bacterial meningitis (Tuomanen et al, 1989 J. Exp. Med. 170, 959-968), vascular injury and death in endotoxic shock (Thomas et al, 1991 FASEB J., 5, A509) and indomethacin-induced gastric injury (Wallace et al, 1991 Gastroenterology 100, 878-883).

Monoclonal antibodies directed to the CD11b subunit have been reported by Todd, R. F. et al., U.S. Pat. No. 4,840,793 (Jun. 20, 1989), Todd, R. F. et al., U.S. Pat. No. 4,935,234 (Jun. 19, 1990), Schlossman, S. F. et al., U.S. Pat. No. 5,019,648 (May 28, 1991) and Rusche, J. R. et al., PCT Application No. WO 92/11870 (Jul. 23, 1992). Monoclonal antibodies directed to CD18 subunit have been reported by Arfors, K. E., U.S. Pat. No. 4,797,277 (Jan. 10, 1989), Wright, S. D. et al., European Patent Application No. EP 0346078 (Dec. 13, 1989), Law, M. et al., European Patent Application No. EP 0438312 (Jul. 24, 1991), Law, M. et al., European Patent Application No. EP 0440351 (Aug. 7, 1991), Wright, S. D. et al., U.S. Pat. No. 5,147,637 (Sep. 15, 1992) and Wegner, C. D. et al., European Patent Application No. EP 0507187 (Oct. 7, 1992).

Antibodies to other adhesive molecules have also been reported to have anti-inflammatory properties.

Monoclonal antibodies that recognise the counter-receptor of CD11a/CD18 and CD11b/CD18, i.e. ICAM-1, have been reported to prolong cardiac allograft survival (Flavin et al, 1991 Transplant. Proc. 23, 533-534) and prevent chemically induced lung inflammation (Barton et al, 1989 J. Immunol. 143, 1278-1282). Furthermore, anti-selectin monoclonal antibodies have also been reported as active in animal models of neutrophil-mediated inflammation. Monoclonal antibodies to L-selectin have been reported to prevent neutrophil migration into inflamed skin (Lewinshon et al., 1987 J. Immunol. 138, 4313) and inflamed ascites (Jutila et al., 1989 J. Immunol. 143, 3318; Watson et al, 1991 Nature 349, 164). Reports have also described inhibition of neutrophil influx into inflamed lung tissue by anti E-selectin monoclonal antibodies (Mulligan et al., 1991 J. Clin. Invest. 88, 1396; Gundel et al., 1991 J. Clin. Invest. 88, 1407). While monoclonal antibodies to adhesive proteins have demonstrated the feasibility of using neutrophil adhesion inhibitors as anti-inflammatory agents, their utility as therapeutics requires further evaluation.

Soluble adhesive receptors obtained by genetic engineering have been proposed as anti-inflammatory compounds. Soluble receptors, in which the transmembrane and intracellular domains have been deleted by recombinant DNA technology, have been tested as inhibitors of neutrophil adhesion to endothelial cells. The functional use of recombinant soluble adhesive molecules has been reported using CD11b/CD18 (Dana et al., 1991 Proc. Natl. Acad. Sci.(USA) 88, 3106-3110) and L-selectin (Watson et al., 1991).

Recently, a new class of anti-leukocyte compounds collectively termed “leumedins” has been reported. These compounds have been reported to block the recruitment in vivo of T-lymphocytes and neutrophils into inflammatory lesions. The mechanism of action of the leumedins is unclear, but there is evidence that they do not function by blocking neutrophil activation (Burch et al., 1991 Proc. Natl. Acad. Sci.(USA) 88, 355). It remains to be determined if leumedins block neutrophil infiltration by direct interference with adhesive molecules.

It has been suggested that parasites survive in their host by modulating host immunity and inflammatory response though the mechanisms by which this occurs remains unclear (Leid, W. S., 1987, Veterinary Parasitology, 25: 147). In this regard, parasite-induced immunosuppression in rodent models has been proposed (Soulsby et al., 1987, Immunol Lett. 16, 315-320). The various aspects of the modulation of host immunity by helminth parasites to evade immunological attack have recently been reviewed. See Maizels et al. (1993), Nature, 365:797-805.

Various parasites have been reported to have an affect on neutrophils of their host. For example, a protein isolated from the cestode, Taenia taeniaeformis, has been reported to inhibit chemotaxis and chemokinesis of equine neutrophils, as well as inhibit neutrophil aggregation (C. Suquet et al., 1984, Int'l J. Parasitol., 14: 165; Leid, R. W. et al., 1987, Parasite Immunology, 1: 195; and Leid, R. W. et al., 1987, Int'l J. Parasitol., 17: 1349). Peritoneal neutrophils from mice infected with the cestode, Echinococcus multiocularis, have been reported to lose their ability to migrate toward parasite antigens and non-specific chemoattractants with increasing time of infection (Alkarmi, T. et al., Exptl. Parasitol., 1989, 69: 16). The nematode, Trichinella spiralis, has been reported to either excrete and/or secrete factors which inhibit chemotaxis and p-nitroblue tetrazolium reduction (i.e. release of oxidative metabolites) but enhance chemokinesis of human neutrophils (Bruschi, F. et al., 1989, Wiadomosci Parazytologiczne, 35: 391). The sera of humans infected with the nematode, Trichinella spiralis, have been reported to inhibit leukocyte chemotaxis and phagocytosis (Bruschi, F. et al., 1990, J. Parasitol., 76: 577). The saliva of the tick, Ixodes dammini, has been reported to inhibit neutrophil function (Ribeiro et al, 1990, Exp. Parasitol., 70, 382). A protein secreted by the cestode, Echinococcus granulosus, has been reported to inhibit human neutrophil chemotaxis (Shepard, J. C. et al., 1991, Mol. Biochem. Parasitol., 44: 81).

Another component of the host defence mechanism against invading pathogens is eosinophils. Functionally, eosinophils are similar to neutrophils in that both cell types have the ability to phagocytose and to release compounds that are either directly or indirectly toxic to pathogenic organisms. Eosinophils are distinguished from neutrophils by their morphologic features, constituents, products and associations with specific diseases. Although eosinophils have been reported to be capable of killing bacteria in vitro, this class of leukocyte alone is not believed sufficient to defend against bacterial infections in vivo. Instead, it is thought that eosinophils afford primary defence against large organisms such as helminthic parasites (Butterworth A E, 1984; Adv. Parasitol. 23:143-235). Also, it is widely held that eosinophils can play a major role in certain inflammatory diseases.

Specifically, substances released from eosinophils that are known collectively as cationic granule proteins, including major basic protein, eosinophil cationic protein and eosinophil-derived neurotoxin, have been implicated in asthma (Gleich G J and Adolphson, C R, 1986; Adv. Immunol. 39:177-253), inflammatory bowel disease (Hällren, R, 1989; Am. J. Med. 86:56-64) and atopic dermatitis (Tsuda, S, et al, 1992; J. Dermatol. 19:208-213). Moreover, other eosinophil products such as superoxide anions, hydroxyl radicals and singlet oxygen may also be involved in damage to host tissue in inflammatory disease states (Petreccia, D C et al, 1987, J. Leukoc. Biol. 41:283-288; Kanofsky, J R et al, 1988; J. Biol. Chem. 263:9692-9696).

An early step in eosinophil-mediated inflammatory disease is believed to be the movement of eosinophils from the vascular compartment to tissue. The first step in this extravasation process is reported to be the adherence of eosinophils to the luminal surface of the vascular endothelium. Although mechanisms of eosinophil-endothelial cell adhesion are not as well defined as those involving adhesion by neutrophils, it is reported that members of the CD11/CD18 family of integrins on the surface of the eosinophil are involved in eosinophil-endothelial adhesion (Lamas, A M, et al, 1988; J. Immunol. 140:1500; Walsh, G M, et al, 1990; Immunology 71:258), and it is reported that the endothelial cell counter-receptor is likely ICAM-1 (Wegner, C D, et al, 1990; Science 247:456-459). A second integrin known as VLA-4 (very late antigen-4; a4b1) that is present on eosinophils, lymphocytes and monocytes but not neutrophils, is thought to contribute to eosinophil adherence by binding to the VCAM-1 (vascular cell adhesion molecule-1) that is expressed on the surface of endothelial cells (Dobrina, A, et al, 1991, J. Clin. Invest. 88:20). IL-1 treatment of the endothelial cell monolayers has been reported to induce an increased adhesiveness for human basophils, eosinophils and neutrophils but treatment of these endothelial cells with an antibody directed to VACM-1 was reported to inhibit both basophil and eosinophil adhesion but not neutrophil adhesion. It has also been reported that monoclonal antibodies against VCAM-1 inhibit lymphocyte and monocyte cell adhesion to stimulated endothelium (Carlos et al. (1990), Blood, 76:965-970; Rice et al., J. Exp. Med. (1990), 171:1369- 1374) but not to neutrophils.

Approaches to the treatment of eosinophil-mediated inflammation have been similar to those adopted for neutrophil-mediated disease. For example, potential therapeutics under investigation for eosinophil-mediated inflammation include glucocorticoids (Evans, P M, et al, 1993, J. Allergy Clin. Immunol. 91:643-650). As is the case for other agents that have been reported to modulate neutrophil function, these agents have been found to be sub-optimal in that they are relatively non-specific and toxic. A second approach to anti-eosinophil therapy has been the use of compounds that directly inhibit the adhesion of eosinophils to vascular endothelium. It has been reported that in animal models of asthma, monoclonal antibody against ICAM-1 blocks eosinophil infiltration into tissues (Wegner et al. (1990), Science, 247:456-459). ICAM-1 and functional derivatives thereof have been proposed as anti-inflammatory agents (Anderson et al., European Patent Application No. EP 0314863 (Apr. 29, 1988); Wegner et al., PCT Application No. WO 90/10453 (Sep. 20, 1990).

However, there remains a need for potent, highly specific inhibitors of neutrophil function, in particular, adhesion to vascular endothelium, as a treatment for abnormal granulocyte-mediated inflammation.

PCT Application Nos. WO 93/23063 (filed May 11, 1993; published Nov. 25, 1993) and WO 94/14973 (filed Dec. 23, 1993; published Jul. 7, 1994) (which published patent applications are incorporated herein by reference) describe potent and specific inhibitors of neutrophil and eosinophil activity (in particular the adhesion of these granulocytes to vascular endothelial cells) derivable from parasitic worms, specifically hookworms (such as Ancylostoma caninum) and related species.

The potent and specific inhibitors of neutrophil and eosinophil activity described in WO 93/23063 and WO 94/14973 (which are incorporated herein by reference) include Neutrophil Inhibitory Factor (NIF); variants, fragments, homologues, analogues and derivatives of NIF; recombinant NIF (rNIF); variants, fragments, homologues, analogues and derivatives of rNIF; NIF mimics; variants, fragments, homologues, analogues and derivatives of NIF mimics; NIF-like proteins; and variants, fragments, homologues, analogues and derivatives of NIF-like proteins. Such molecules (collectively known as “NIFs”) represent a pioneering step toward the development of a new generation of anti-inflammatory therapeutic products. This discovery will enable therapy for inflammatory disease based entirely on specific inhibition of the inflammatory response. The therapeutic advantages of this novel approach are realised through the specificity of NIF compared to current clinical treatment modalities such as steroids, catecholamines, prostaglandins, and non-steroidal anti-inflammatory agents. The currently used therapeutic agents demonstrate poor efficacy and multiple adverse reactions due to generalised systemic effects that non-specifically target numerous biological processes in addition to the inflammatory process.

Nonetheless, the existence of this extensive panel of anti-inflammatory agents, although sub-optimal, and the total funds expended by the pharmaceutical industry in research in this area, point to significant medical needs for effective anti-inflammatory agents and suggests that the novel and highly specific NIFs described in WO 93/23063 and WO 94/14973 have important applications.

Brain and spinal cord injury caused by stroke, trauma or hypoxia often results in lifelong disability and premature death. The cause of disability and death is the disruption of function and death of neurons and other cells in the central nervous system. Therefore, a clear benefit is anticipated from therapies that reduce or prevent neuronal dysfunction and death after ischaemic, hypoxic or traumatic central nervous system (CNS) insult.

As noted above, the inflammatory response may result in clinical syndromes ranging from debilitating arthritis and asthma to life threatening shock. In view of the severity of these disorders, the vast number of individuals afflicted therewith and the lack of suitable therapeutic intervention, the need for a breakthrough therapy represents a long felt need which has not been met.

Further, in view of the myriad conditions associated with undesired and/or abnormal inflammatory conditions which appear to be associated with neutrophil activity, there remains a need for potent, highly specific inhibitors of neutrophil function, in particular, adhesion to vascular endothelium, as a treatment for abnormal neutrophil-mediated inflammation.

The present invention is believed to fulfil this need by disclosing, inter alia, a combination therapy involving use of a potent and specific inhibitor of neutrophil activity (in particular the adhesion of neutrophils to vascular endothelial cells) derivable from (i) parasitic worms, specifically hookworms (such as Ancylostoma caninum) and related species or (ii) synthetically (i.e. recombinantly).

Such a combination therapy employs the therapeutic benefits that may be gained by treating traumatic brain injury, stroke, or hypoxic brain injury with NIF in combination with other types of compounds. These include compounds that also protect neurons from toxic insult, inhibit the inflammatory reaction after brain damage and/or promote cerebral reperfusion. Although necrosis is a principal cause of the neuronal dysfunction and death that occurs after CNS insult, additional mechanisms also participate (Dirnagl et al Trends Neurosci. 1999;22;391-397). By reducing the pathological consequences of these additional mechanisms, the overall benefit of the therapeutic intervention may be increased. Furthermore, inhibiting multiple pathological processes may provide an unexpected therapeutic benefit over and above that which may be achievable alone with the use of NIF.

During the course of an ischaemic, hypoxic, or traumatic injury to the CNS a number of toxic products are formed which can further damage brain cells injured by the primary pathological process or produce damage in cells that otherwise escape damage from the primary insult. These toxins include, but are not limited to: nitric oxide (NO); other reactive oxygen and nitrogen intermediates such as superoxide and peroxynitrite; lipid peroxides; TNFα, IL-1 and other interleukins, cytokines or chemokines; cyclooxygenase and lipoxygenase derivatives and other fatty acid mediators such as leukotrienes, glutamate and prostaglandins; and hydrogen ions Barone and Feuerstein 1999 J. Cereb. Blood Flow Metab.;19:819-834 and Lee et al 1999 Nature;399:A7-A14). Inhibiting the formation, action or accelerating the removal of these toxins may protect CNS cells from damage during an ischaemic, hypoxic or traumatic injury. Furthermore, inhibiting the formation, action or accelerating the removal of these toxins may have additional benefits when combined with the benefits of inhibiting neutrophil function via NIF.

Examples of compounds that inhibit the formation or action of these toxins, or accelerate their removal include, but are not limited to, antioxidants, sodium channel antagonists, nitric oxide synthase (NOS) inhibitors, potassium channel openers, NMDA receptor antagonists, NMDA glycine site receptor antagonists, AMPA (2-amino-3-(methyl-3-hydroxyisoxazol-4-yl)propanoic acid)/kainate receptor antagonists, calcium channel antagonists, GABA_{A} receptor modulators (e.g., GABA_{A} receptor agonists), selective serotonin reuptake inhibitors (SSRIs), 5-HT_{1A} agonists and anti-inflammatory agents.

The formation and release of many of the toxins listed above are triggered by physiological signalling mechanisms that become pathologically activated by ischaemic, hypoxic or traumatic CNS injury. Activation of these signalling mechanisms can also result in cellular depolarisation. This depolarisation may disrupt cellular ionic homeostasis, accelerate the rate of energy utilisation as the cell strives to maintain homeostasis, and/or further accelerate the rate of formation and release of toxins. Thus, inhibition of these signalling mechanisms during ischaemic, hypoxic or traumatic CNS injury may reduce the degree of cellular dysfunction and death. Furthermore, inhibiting these signalling mechanisms may have additional benefits when combined with the benefits of inhibiting neutrophil function via NIF. These signalling mechanisms include, but are not limited to: N-methyl-D-aspartate (NMDA) receptors; other excitatory amino acid (EM) receptors such as AMPA, kainate, or metabotropic receptors; other ligand-gated ion channels which promote depolarisation and/or toxin release; voltage gated calcium channels including those of the L-, P-, Q/R-, N-, or T- types; voltage gated sodium channels. Examples of compounds that inhibit these signalling pathways include, but are not limited to, AMPA/kainate receptor antagonists, sodium channel antagonists and calcium channel antagonists.

Another approach to inhibiting cellular depolarisation caused by ischaemic, hypoxic or traumatic CNS injury and the resultant deleterious effects is to activate signalling pathways that oppose those causing depolarisation. Again, activating these signalling mechanisms may have additional benefits when combined with the benefits of inhibiting neutrophil function via NIF. These signalling mechanisms include, but are not limited to: GABA_{A} receptor activation; voltage- or ligand-gated potassium channel activation; voltage- or ligand-gated chloride channel activation. Examples of compounds that activate these signalling pathways include, but are not limited to, potassium channel openers and GABA_{A} receptor agonists.

Excessive cellular depolarisation and the loss of ionic homeostasis can lead to the loss in the ability of a cell to maintain physical integrity and cellular death ensues by a process often termed necrotic cell death. However, ischaemic, hypoxic or traumatic CNS injury can also induce in many cells the activation of another mechanism causing a programmed cell death that is termed apoptosis. The relationship between necrotic and apoptotic cell death is not fully understood and in pathological conditions such as ischaemic, hypoxic or traumatic CNS injury both necrotic and apoptotic mechanisms leading ultimately toward cell death may be at play (Lee et al 1999 Nature;399:A7-A14; Dirnagl et al Trends Neurosci. 1999;22;391-397). Regardless of the specifics of this interrelationship, it has been suggested that inhibition of apoptotic mechanism of cell death may have a therapeutic benefit in ischaemic, hypoxic or traumatic CNS injury. Inhibiting apoptosis during ischaemic, hypoxic or traumatic CNS injury may have additional benefits when combined with the benefits of inhibiting neutrophil function via NIF. Apoptotic mechanisms include, but are not limited to: activation of FAS/TNFα/p75 receptors; activation of caspases including caspase-1 to caspase-9; activation of NFκB; activation of the JNK and/or p38 kinase signalling cascades (Schulz et al Ann. Neurol. 1999;45:421-429, Barone and Feuerstein 1999 J. Cereb. Blood. Flow Metab. 19:819-834); inhibition of mitochondrial disruption and the activation of the mitochondrial permeability transition pore; and activation of intracellular proteases such as the calpains. Examples of compounds that inhibit these apoptotic mechanisms include, but are not limited to, caspase inhibitors and inhibitors of the other enzymes mentioned above as mediators of apoptotic mechanisms.

Cells in the CNS are highly dependent on cell-to-cell interactions and interaction with the extracellular matrix for survival and proper function. However, during ischaemic, hypoxic, or traumatic CNS insult these interactions are often disrupted and this can lead directly to or contribute to cellular dysfunction and death. Thus, therapies that maintain cell-to-cell and cell-to-extracellular matrix interaction during ischaemic, hypoxic or traumatic CNS insult are expected to reduce dysfunction and cell death. Furthermore, therapies that maintain cell-to-cell and cell-to-extracellular matrix interaction during ischaemic, hypoxic or traumatic CNS injury may have additional benefits when combined with the benefits of inhibiting neutrophil function via NIF. Mechanisms that contribute to the disruption of cell-to-cell and cell-to-extracellular matrix interaction during ischaemic, hypoxic or traumatic CNS insult include, but are not limited to: the activation of proteases which degrade the extracellular matrix. These include, but are not limited to, matrix metalloproteases such as MMP-1 to MMP-13. Examples of compounds that inhibit these enzymes include, but are not limited to those referred to in the following patents and patent applications: U.S. Pat. No. 5,861,510, issued Jan. 19, 1999; European Patent Application EP 0606046, published Jul. 13, 1994; European Patent Application EP 0935963, published Aug. 18, 1999; PCT Patent Application WO 98/34918, published Aug. 13, 1998; PCT Patent Applications WO 98/08825 and WO 98/08815, both published Mar. 5, 1998; PCT Patent Application WO 98/03516, published Jan. 29, 1998; and PCT Patent Application WO 98/33768, published Aug. 6, 1998.

CNS ischaemia, hypoxia, or trauma leads to an inflammatory response mediated by various components of the innate and adaptive immune system. Because of the nature of the CNS and its unique relationship to the immune system, the immune system activation caused by CNS ischaemia, hypoxia, or trauma can exacerbate cellular dysfunction and death. The mechanisms whereby immune activation exacerbates CNS injury are many-fold. Immune cells resident to the CNS, such as astrocytes and microglia, are activated following CNS injury. Furthermore, peripheral immune cells are recruited to enter the CNS and also become activated. These cells include monocytes/macrophages, neutrophils, and T-lymphocytes. Recruitment and activation of these peripheral immune cells into the CNS after injury involves many of the same mechanisms by which these cells are recruited to and activated by injured tissue outside the CNS. The cells within the area of tissue injury and the vasculature around the site of injury begin to produce proteins that signal to immune cells circulating in the blood stream. These cells then adhere to the vascular epithelium and enter the area in and around the damaged tissue. These activated immune cells then promote many of the deleterious events listed above, including release of a variety of toxins and disruption of cell-to-cell and cell-to-extracellular matrix interactions.

Thus, inhibition of immune cell recruitment, adherence to the vasculature, activation, and formation and release of toxins and proteases in response to CNS ischaemia, hypoxia, or trauma is hypothesised to reduce the cellular dysfunction and death caused by these CNS insults. Inhibiting immune cell recruitment, activation, and formation and release of toxins and proteases during ischaemic, hypoxic or traumatic CNS injury may have additional benefits when combined with the benefits of inhibiting neutrophil function via NIF. Compounds that inhibit immune cell recruitment include, but are not limited to, antagonists to a wide variety of cytokine and chemokine receptors. Compounds other than NIF, that inhibit immune cell adherence to the vasculature include, but are not limited to, antibodies to a variety of cell adhesion molecules. Compounds other than NIF, that inhibit immune cell activation include, but are not limited to, antagonists to a wide variety of cytokine and chemokine receptors, antibodies to a variety of cell adhesion molecules, antagonists of intracellular enzymes involved in transducing the activating signal into a cellular response such as antagonists of COX and COX2, various protein ser/thr and tyr kinases and intracellular proteases. Recruitment, adherence, and activation of CNS resident and peripheral immune cells can also be inhibited by the activation of cell signalling pathways that oppose this activation. Compounds that activate such signalling pathways include, but are not limited to, PPARγ activators.

In the case of ischaemic (thrombotic or embolic) stroke, it has been observed that administration of agents that degrade thrombi and emboli can have a beneficial effect on patient survival, recovery and/or function. The mechanism of action of these agents is to promote the reperfusion of ischaemic tissue. It is suggested here that the benefits of reperfusion using agents that promote reperfusion following thrombotic or embolic stroke may have additional benefits when combined with the benefits of inhibiting neutrophil function via NIF in these conditions. Compounds that promote reperfusion following thrombotic or embolic stroke include, but are not limited to, tissue plasminogen activator (t-PA) and variants thereof, urokinase, pro-urokinase and streptokinase.

Over-promotion of reperfusion can result in reperfusion injury—both spontaneous or via the use of thrombolytics/fibrinolytics. Hence, a subtle balance must be struck between the anti-neutrophilic action of NIF and the pro-reperfusion action of, for example, thrombolytic/fibrinolytics, such as t-PA or its variants.

The present invention strikes such a balance and provides, inter alia, a combination therapy that utilises the combined benefits of at least one NIF and at least one other neuroprotective or thrombolytic/fibrinolytic agent for the treatment of pathophysiological conditions involving neutrophils.

### SUMMARY OF THE INVENTION

According to a first aspect of the present invention, there is provided the use of a combination of at least one Neutrophil Inhibitory Factor (NIF) and at least one other neuroprotective or thrombolytic/fibrinolytic agent or a pharmaceutically acceptable salt thereof in the manufacture of a medicament for the treatment of pathophysiological conditions involving neutrophils.

According to a second aspect of the present invention, there is provided a method of treating pathophysiological conditions involving neutrophils, comprising administering to a subject in need of said treatment, either simultaneously, separately or sequentially, a combination of:

(a) at least one Neutrophil Inhibitory Factor (NIF); and

(b) at least one other neuroprotective or thrombolytic/fibrinolytic agent or a pharmaceutically acceptable salt thereof;

wherein the two or more agents of (a) or (b) above are present in amounts that render the combination of said two or more agents effective in treating pathophysiological conditions involving neutrophils.

According to a third aspect of the present invention, there is provided a pharmaceutical composition comprising:

(a) at least one Neutrophil Inhibitory Factor (NIF);

(b) at least one other neuroprotective or thrombolytic/fibrinolytic agent or a pharmaceutically acceptable salt thereof; and optionally

(c) a pharmaceutically acceptable carrier, diluent, excipient or adjuvant.

According to a fourth aspect of the present invention, there is provided a process for preparing the pharmaceutical composition described above, comprising the steps of:

(a) performing an assay to identify one or more agents that is/are, or has/have the capability of acting as, Neutrophil Inhibitory Factor (NIF);

(b) admixing one or more of said agent(s) with one or more other neuroprotective or thrombolytic/fibrinolytic agent(s); and optionally admixing

(c) a pharmaceutically acceptable carrier, diluent, excipient or adjuvant therewith.

Preferably, said process also includes the subsequent step of:

(d) administering said pharmaceutical composition to a subject in need of the same.

According to a fifth aspect of the present invention, there are provided products containing:

(a) at least one Neutrophil Inhibitory Factor (NIF); and

(b) at least one other neuroprotective or thrombolytic/fibrinolytic agent or a pharmaceutically acceptable salt thereof;

as a combined preparation for simultaneous, separate or sequential use in treating pathophysiological conditions involving neutrophils.

Preferred embodiments in relation to each of the above aspects of the present invention are set out as follows:

(i) Preferably, said Neutrophil Inhibitory Factor (NIF) has the amino acid sequence as set out in SEQ ID NO: 3 or 4 or a fragment, variant, homologue, derivative or analogue thereof. More preferably, said Neutrophil Inhibitory Factor (NIF) is UK-279,276.

(ii) Preferably, said pathophysiological condition involving neutrophils is ischaemic damage and/or reperfusion injury. More preferably, said ischaemic damage and/or reperfusion injury is stroke, traumatic head injury, post-ischaemic-reperfusion injury, post-ischaemic cerebral inflammation or ischaemia-reperfusion injury following myocardial infarction. Most preferably, said pathophysiological condition involving neutrophils is stroke. In a preferred embodiment of the present invention said stroke is acute stroke. More preferably, said stroke is ischaemic stroke. Most preferably, said ischaemic stroke is thrombotic or embolic stroke. Alternatively, said stroke can be haemorrhagic stroke and said at least one other neuroprotective or thrombolytic/fibrinolytic agent is a neuroprotective agent.

(iii) Preferably, said neuroprotective or thrombolytic/fibrinolytic agent(s) is/are any one or more of a plasminogen activator, urokinase, pro-urokinase, streptokinase, p-anisoylated plasminogen streptokinase activator complex (APSAC), urokinase plasminogen activator (uPA), a MMP inhibitor, a sodium channel antagonist, a nitric oxide synthase (NOS) inhibitor, a NMDA receptor antagonist, a NMDA glycine site receptor antagonist, a potassium channel opener, an AMPA/kainate receptor antagonist, a calcium channel antagonist, a GABA_{A} receptor modulator, a GABA_{A} receptor agonist, an SSRI, a 5-HT_{1A} agonist or an anti-inflammatory agent. More preferably, said plasminogen activator is tissue plasminogen activator (t-PA) or variants thereof or Desmoteplase. Preferably, said variants of tissue plasminogen activator (t-PA) are Alteplase, Monteplase, Reteplase, Lanoteplase, Duteplase and Tenecteplase. Most preferably, said variant of tissue plasminogen activator (t-PA) is Alteplase, Monteplase or Tenecteplase.

(iv) Preferably, said pathophysiological condition involving neutrophils is stroke and the therapeutic time window of administration of said at least one other neuroprotective or thrombolytic/fibrinolytic agent is 0 to > about 3 h from onset of stroke. Other therapeutic time windows of administration of said at least one other neuroprotective or thrombolytic/fibrinolytic agent contemplated by the present invention include: 0 to ≦ about 3 h; 0 to ≦3 h; 0 to ≧ about 3 h; 0 to ≧3 h; 0 to >3 h; 0 to ≦ about 4 h; 0 to ≦4 h; 0 to ≧ about 4 h; 0 to ≧4 h; 0 to >4 h; 0 to ≦ about 6 h; 0 to ≦6 h; 0 to ≧ about 6 h; 0 to ≧6 h; 0 to >6 h; 0 to ≦ about 8 h; 0 to ≦8 h; 0 to ≧ about 8 h; 0 to ≧8 h; 0 to >8 h; 0 to ≦ about 10 h; 0 to ≦0 h; 0 to ≧ about 10 h; 0 to ≧10 h; 0 to >10 h; 0 to ≦ about 12 h; and 0 to ≦12 h. More preferably, said therapeutic time window of administration of said at least one other neuroprotective or thrombolytic/fibrinolytic agent is 0 to ≦ about 6 h from onset of stroke, most preferably approximately 4 h to 6 h from onset of stroke. Preferably, at least one other neuroprotective or thrombolytic/fibrinolytic agent is a plasminogen activator, preferably tissue plasminogen activator (t-PA) or variants thereof or Desmoteplase. More preferably, said variants of tissue plasminogen activator (t-PA) are Alteplase, Monteplase, Reteplase, Lanoteplase, Duteplase and Tenecteplase. Most preferably, said variant of tissue plasminogen activator (t-PA) is Alteplase, Monteplase or Tenecteplase.

The present invention will now be described, by way of example only, with reference to the accompanying Figures and Sequence Listing, in which:

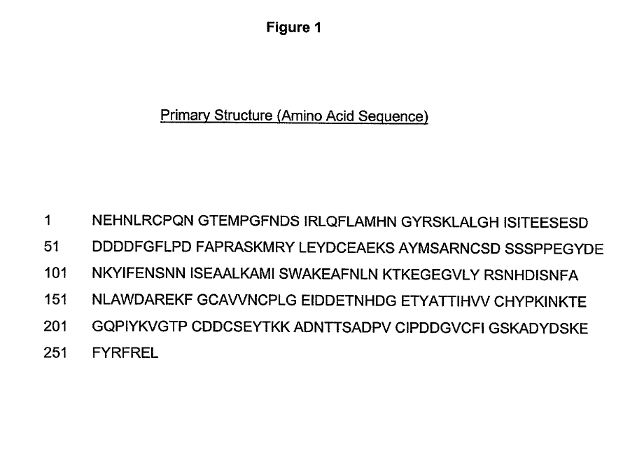

FIG. 1 shows the primary structure (amino acid sequence) of NIF.

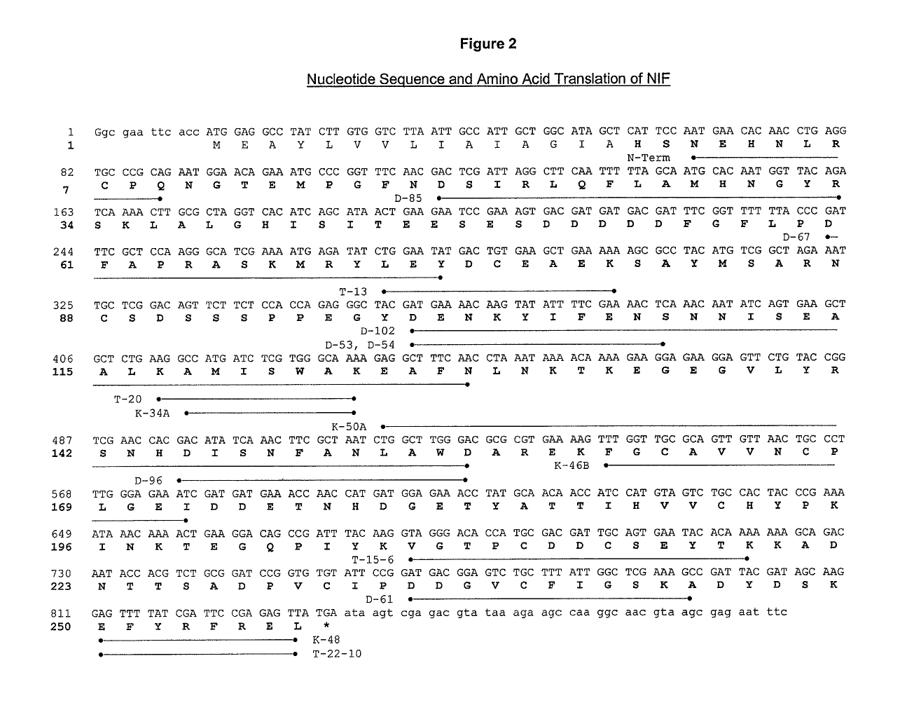

FIG. 2 shows the nucleotide sequence and amino acid translation of NIF.

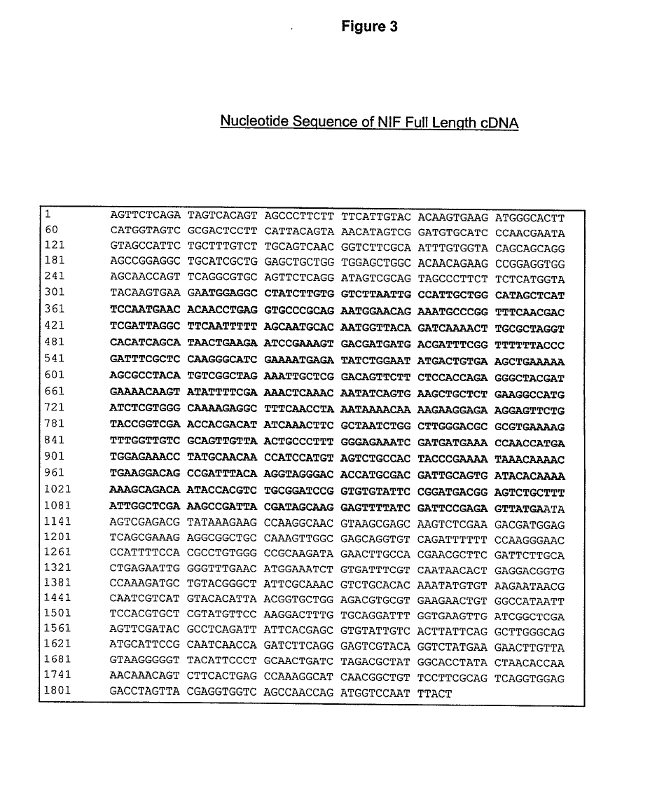

FIG. 3 shows the nucleotide sequence of NIF full-length cDNA.

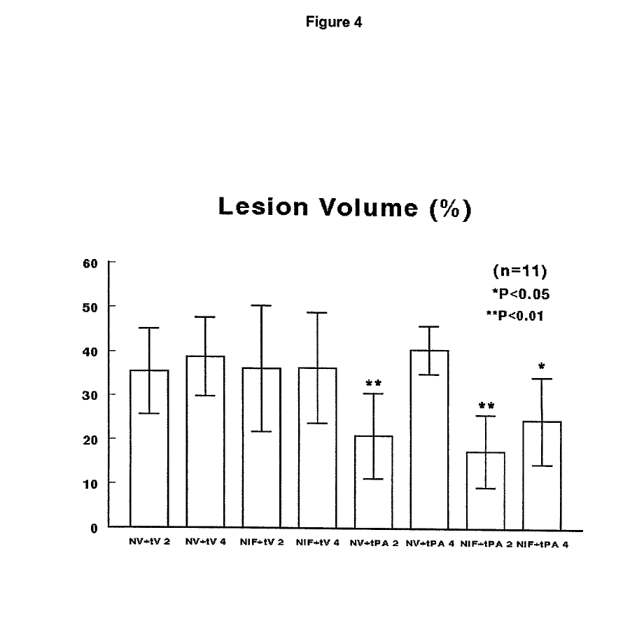

FIG. 4 shows the effect of treatment groups on infarct volume (abbreviations for relevant treatment groups and explanations are presented in Table 1 in Example 1).

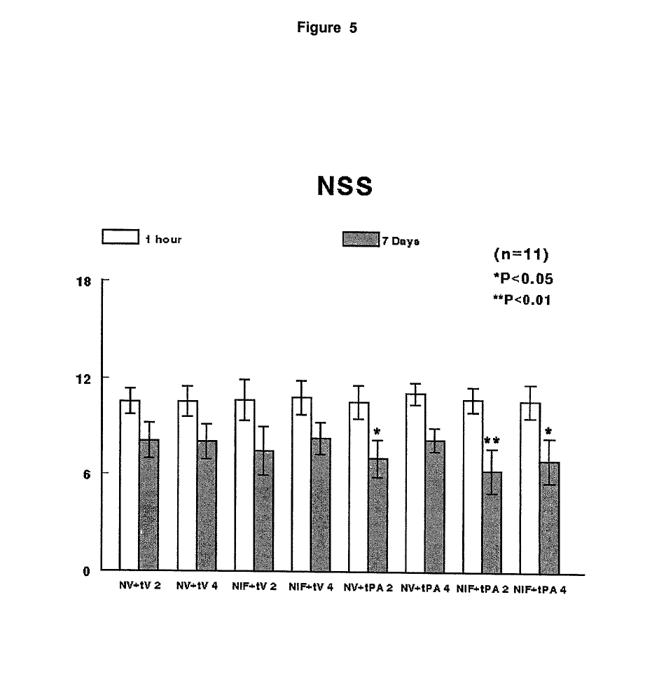

FIG. 5 shows the effect of treatment groups on neurological severity score (NSS) at 1 h and 7 days after embolization (abbreviations for relevant treatment groups and explanations are presented in Table 1 in Example 1).

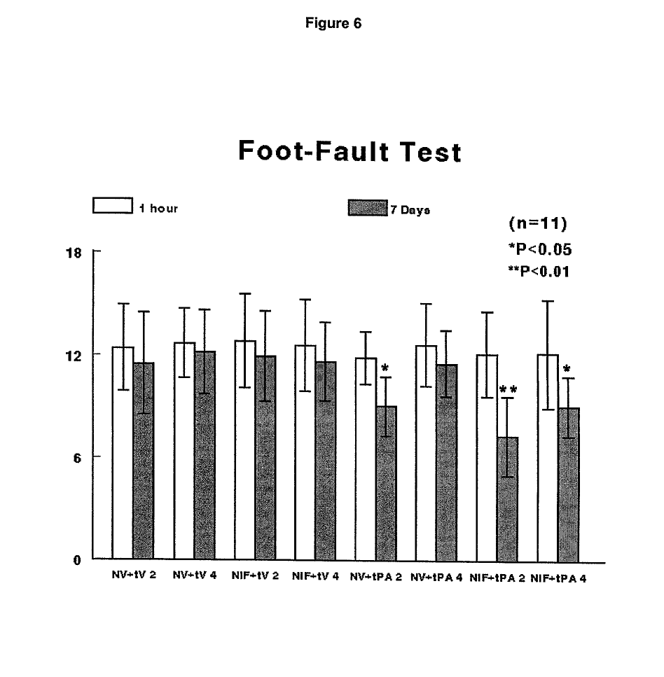

FIG. 6 shows the effect of treatment groups on foot-fault test at 1 h and 7 days after embolization (abbreviations for relevant treatment groups and explanations are presented in Table 1 in Example 1).

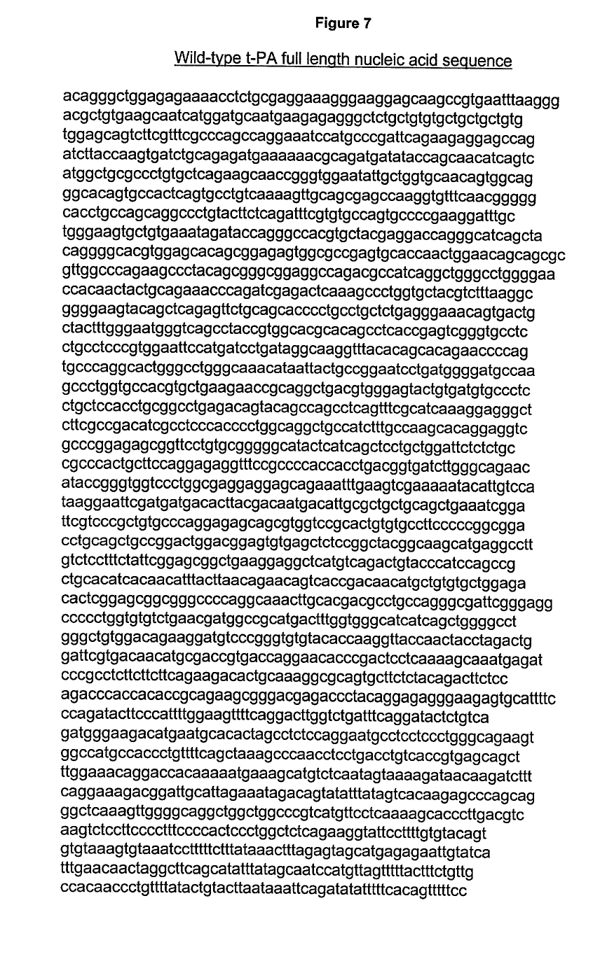

FIG. 7 shows wild-type t-PA full-length nucleic acid sequence.

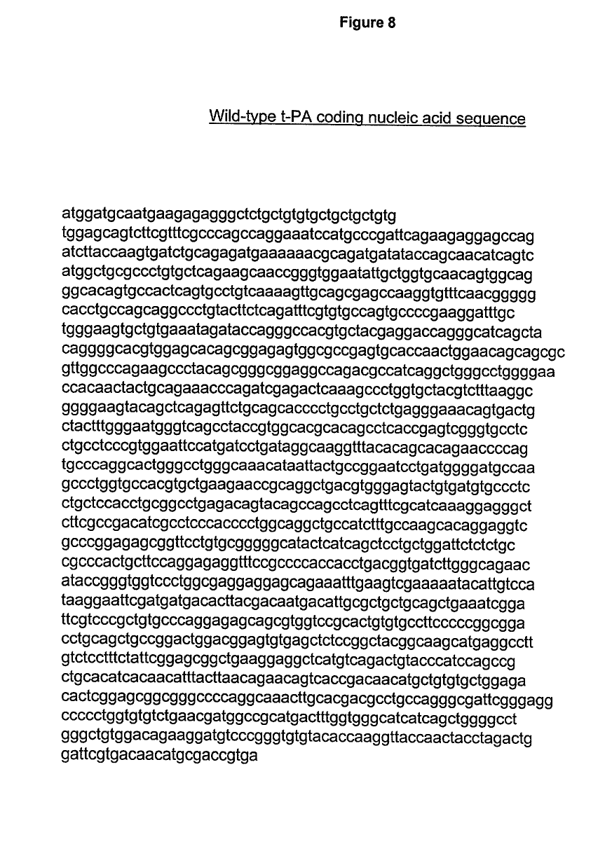

FIG. 8 shows wild-type t-PA coding nucleic acid sequence.

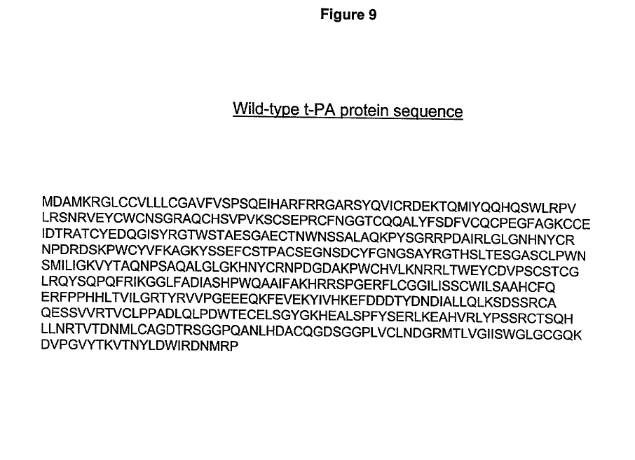

FIG. 9 shows wild-type t-PA protein sequence.

SEQ ID NO: 1 shows the nucleotide sequence of NIF full-length cDNA.

SEQ ID NO: 2 shows the nucleotide sequence coding for the primary structure of NIF.

SEQ ID NO: 3 shows the amino acid translation of the nucleotide sequence coding for the primary structure of NIF.

SEQ ID NO: 4 shows the primary structure (amino acid sequence) of NIF.

SEQ ID NO: 5 shows wild-type t-PA full-length nucleic acid sequence.

SEQ ID NO: 6 shows wild-type t-PA coding nucleic acid sequence.

SEQ ID NO: 7 shows wild-type t-PA protein sequence.

### Detailed Description of Invention

Neutrophil Inhibitory Factor

Neutrophil Inhibitory Factors (NIFs) are proteins that are specific inhibitors of neutrophil activity, in particular of the adhesion of neutrophils to vascular endothelial cells, and which are derivable from e.g. hookworms and related species. NIFs can be isolated from natural sources or made by recombinant methods, and which, when isolated from parasitic worms, are glycoproteins. NIFs are not members of the integrin or selectin families of proteins and also are not members of the immunoglobulin superfamily of adhesive proteins.

Neutrophils are a subset of the class of cells known as granulocytes, which are members of a subclass of cells known as leukocytes. Neutrophils are an essential component of the host defence system against microbial invasion. In response to soluble inflammatory mediators released by cells at the site of injury, neutrophils migrate into tissue from the bloodstream by crossing the blood vessel wall. At the site of injury, activated neutrophils kill foreign cells either by phagocytosis and/or by the release of cytotoxic compounds, such as oxidants, proteases and cytokines. Despite their importance in fighting infection, neutrophils themselves can promote tissue damage. During an abnormal inflammatory response, neutrophils can cause significant tissue damage by releasing toxic substances at the vascular wall or in uninjured tissue. Alternatively, neutrophils that adhere to the capillary wall or clump together in venules may produce tissue damage by ischaemia (“no reflow” phenomenon). Such abnormal inflammatory responses have been implicated in the pathogenesis of a variety of clinical disorders including adult respiratory distress syndrome (ARDS); ischaemia-reperfusion injury following myocardial infarction, shock, stroke, and organ transplantation; acute and chronic allograft rejection; vasculitis; sepsis; rheumatoid arthritis; and inflammatory skin diseases (Harlan et al., 1990 Immunol. Rev. 114, 5).

Neutrophil adhesion at the site of inflammation is believed to involve at least two discrete cell-cell interactive events. Initially, vascular endothelium adjacent to inflamed tissue becomes adhesive to activated neutrophils; neutrophils interact with the endothelium via low affinity adhesive mechanisms in a process known as “rolling”. In the second adhesive step, rolling neutrophils bind more tightly to vascular endothelial cells and migrate from the blood vessel into the tissue.

The inhibition of neutrophil activity by the NIFs of the present invention includes but is not limited to inhibition of one or more of the following activities by neutrophils: release of hydrogen peroxide, release of superoxide anion, release of myeloperoxidase, release of elastase, homotypic neutrophil aggregation, adhesion to plastic surfaces, adhesion to vascular endothelial cells, chemotaxis, transmigration across a monolayer of endothelial cells and phagocytosis.

The NIFs of the present invention are preferably further characterised as also having the ability to bind to the CD11b/CD18 receptor and also preferably as having the ability to bind to the I-domain portion of the CD11b/CD18 receptor. The NIFs of the present invention may be preferably further characterised as having eosinophil inhibitory activity.

NIFs are described in greater detail, along with methods of isolating them from natural sources and of cloning them, in WO 93/23063, WO 94/14973, U.S. Pat. No. 5,708,141, which issued on Jan. 13, 1998, U.S. Pat. No. 5,919,900, which issued on Jul. 6, 1999, U.S. Pat. No. 5,747,296, which issued on May 5, 1998, and U.S. Pat. No. 5,789,178, which issued on Aug. 4, 1998 (all of which are incorporated herein by reference). Preferred NIFs for use in the pharmaceutical compositions and methods of the present invention are those that are designated as preferred NIFs in WO 93/23063, WO 94/14973 and U.S. Pat. No. 5,919,900, referred to above (all of which are incorporated herein by reference).

A preferred NIF of the present invention (Pfizer's UK-279,276 compound) is a glycoprotein which, when not glycosylated, has a molecular weight (MW) of about 29,507 Daltons (carboxymethylated UK-279,276) as measured by ESI-MS (Electrospray Ionisation Mass Spectroscopy). This preferred NIF, when glycosylated, has a MW within the range of about 38,342 to 61,377 Daltons (±˜10 Daltons) as measured by MALDI-MS (Matrix Assisted Laser Desorption/Ionisation Mass Spectrometry). This NIF can also be sialylated (i.e. possess sialic acid capped glycan branches). When desialylated, the preferred NIF has an isoelectric point (pI) within the range of about 4.15 to 4.55, including major bands centred at a pI of approximately 4.3 (when determined by isoelectric focusing (IEF)). This experimental value is close to the value of pI 4.66 predicted for the UK-279,276 amino acid sequence. The pI of reduced NIF extends over the range <2.8 to 4.15, indicating a high degree of sialic acid incorporation.

UK-279,276 is a 257 amino acid protein which has the molecular formula C₁₂₅₅H₁₈₉₃N₃₄₁O₄₁₈S₁₅ (deglycosylated) and the primary structure (amino acid sequence) sequence as shown in FIG. 1 (SEQ ID NO: 4) and has been reported in the literature (Moyle M., et al, J. Biol. Chem. 1994, 269, 10008-10015).

The amino acid sequence indicates that there are seven potential N-linked glycosylation sites (Asn₁₀, Asn₁₈, Asn₈₇, Asn₁₁₀, Asn₁₃₀, Asn₁₉₇ and Asn₂₂₃) and ten cysteines with the potential for disulphide bond formation (Cys₇, Cys₇₅, CYS₈₈, CYS₁₆₂, Cys₁₆₇, Cys₁₉₁, Cys₂₁₁, Cys₂₁₄, Cys₂₃₁ and Cys₂₃₈).

The theoretical average mass for the polypeptide backbone of recombinant UK-279,276 is 28,927 Daltons. Given this, UK-279,276 reference standard has an estimated N-linked carbohydrate composition of between 25 and 53%.

UK-279,276 has a relative molecular mass of 28.9 kDa, which undergoes post-translational modification to yield a glycoprotein of relative molecular mass of approximately 38.3 to 61.4 kDa. UK-279,276 is a recombinant glycoprotein derived from a genetically manipulated Chinese Hamster Ovary (CHO) cell line. However, UK-279,276 was first isolated from the canine hookworm Ancylostoma caninum and is known to bond selectively to the CD11b protein on neutrophils, blocking adhesion and activation of those cells that are mediated by this mechanism (see, inter alia, WO 93/23063 and WO 94/14973).

UK-297,276 is therefore a NIF having, inter alia, any one or more of the following characteristics:

(i) Molecular formula of C₁₂₅₅H₁₈₉₃N₃₄₁O₄₁₈S₁₅ (deglycosylated);

(ii) Full-length cDNA nucleotide sequence as shown in SEQ ID NO: 1 (FIG. 3);

(iii) Nucleotide sequence (coding for the primary amino acid structure) as shown in SEQ ID NO: 2 (FIG. 2);

(iv) Amino acid translation of the nucleotide sequence (coding for the primary amino acid structure) as shown in SEQ ID NO: 3 (FIG. 2);

(v) Primary amino acid structure as shown in SEQ ID NO: 4 (FIG. 1);

(vi) Theoretical relative molecular mass of approximately 28.9 kDa (deglycosylated);

(vii) Relative molecular mass of approximately 29.5 kDa (deglycosylated) as measured by ESI-MS;

(viii) Relative molecular mass of approximately 38.3 to 61.4 kDa (glycosylated) as measured by MALDI-MS;

(ix) Theoretical isoelectric point (pi) of 4.66;

(x) Isoelectric point (pI) within the range of about 4.15 to 4.55 (desialylated) as measured by IEF; and

(xi) Isoelectric point (pI) within the range of about <2.8 to 4.15 (reduced) as measured by IEF.

However, for the avoidance of doubt, the term “Neutrophil Inhibitory Factor(s)” or “NIF(s)” refers to any protein or glycoprotein or fragment, variant, homologue, derivative or analogue thereof having neutrophil inhibitory activity (and also eosinophil inhibitory activity or both such activities). Preferably, the NIF employed in the present invention has the amino acid sequence as set out in FIG. 8 (or a fragment, variant, homologue, derivative or analogue thereof) of both WO 93/23063 and WO 94/14973 (which published patent applications are incorporated herein by reference) and FIGS. 1 and 2 (=SEQ ID NO: 4 and 3; or a fragment, variant, homologue, derivative or analogue thereof) of the present application. More preferably, the NIF employed in the present invention is Pfizer's UK-279,276 compound (see above).

For completeness, it should be noted that the term “NIF” encompasses NIF; variants, fragments, homologues, analogues and derivatives of NIF; recombinant NIF (rNIF); variants, fragments, homologues, analogues and derivatives of rNIF; NIF mimics; variants, fragments, homologues, analogues and derivatives of NIF mimics; NIF-like proteins; and variants, fragments, homologues, analogues and derivatives of NIF-like proteins.

The term “NIF mimic” refers to a small molecule, peptide, peptide analogue or protein, which competes with NIF for binding to the CD11b/CD18 receptor or the I-domain portion of the CD11b/CD18 receptor. A “NIF mimic” is also characterised as having neutrophil inhibitory activity, eosinophil inhibitory activity or both such activities.

For the avoidance of doubt, the term “NIF” also includes variants, fragments, homologues, analogues and derivatives of the proteins or glycoproteins described above. Again, specific reference is made to the complete texts of published PCT Applications WO 93/23063 and WO 94/14973, which are incorporated herein by reference.

The terms “variant”, “homologue”, “derivative”, “fragment” or “analogue” in relation to the amino acid sequence of NIF include any substitution of, variation of, modification of, replacement of, deletion of or addition of one (or more) amino acid from or to the sequence providing the resultant protein or (poly)peptide has NIF activity, preferably being at least as biologically active as the polypeptide shown in attached SEQ ID NO: 3 or 4. In particular, the term “homologue” covers homology with respect to structure and/or function. With respect to sequence homology, preferably there is at least 70%, more preferably at least 80%, even more preferably at least 85% homology to the sequence shown in SEQ ID NO: 3 or 4. Preferably there is at least 90%, more preferably at least 95%, most preferably at least 98% homology to the sequence shown in SEQ ID NO: 3 or 4.

Typically, for the variant, homologue, derivative, fragment or analogue of the present invention, the types of amino acid substitutions that could be made should maintain the hydrophobicity/hydrophilicity of the amino acid sequence. Amino acid substitutions may be made, for example from 1, 2 or 3 to 10, 20 or 30 substitutions provided that the modified sequence retains the ability to act as a NIF in accordance with the present invention. Amino acid substitutions may include the use of non-naturally occurring analogues, for example to increase blood plasma half-life of a therapeutically administered polypeptide.

Conservative substitutions may be made, for example, according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As indicated above, proteins of the invention are typically made by recombinant means, for example as described herein, and/or by using synthetic means using techniques well known to the skilled person such as solid phase synthesis. Variants and derivatives of such sequences include fusion proteins, wherein the fusion proteins comprise at least the amino acid sequence of NIF being linked (directly or indirectly) to another amino acid sequence. Preferably the fusion protein partner will not hinder the function of the linked NIF.

The amino acid sequence of NIF may be produced by expression of a nucleotide sequence coding for same in a suitable expression system.

Naturally Occurring

As used herein “naturally occurring” refers to a NIF with an amino acid sequence found in nature.

Isolated/Purified

As used herein, the terms “isolated” and “purified” refer to molecules, either nucleic or amino acid sequences, that are removed from their natural environment and isolated or separated from at least one other component with which they are naturally associated.

Biologically Active

As used herein “biologically active” refers to a NIF—such as a recombinant NIF (rNIF)—having a similar structural function (but not necessarily to the same degree), and/or similar regulatory function (but not necessarily to the same degree), and/or similar biochemical function (but not necessarily to the same degree) and/or immunological activity (but not necessarily to the same degree) of the naturally occurring NIF.

Immunological Activity

As used herein, “immunological activity” is defined as the capability of the natural, recombinant or synthetic NIF or any oligopeptide thereof, to induce a specific immune response in appropriate animals or cells and to bind with specific antibodies.

Derivative

The term “derivative” as used herein in relation to the amino acid sequence includes chemical modification of a NIF. Illustrative of such modifications would be replacement of hydrogen by an alkyl, acyl, or amino group.

Deletion

As used herein a “deletion” is defined as a change in either nucleotide or amino acid sequence in which one or more nucleotides or amino acid residues, respectively, are absent.

Insertion/Addition

As used herein an “insertion” or “addition” is a change in a nucleotide or amino acid sequence, which has resulted in the addition of one or more nucleotides or amino acid residues, respectively, as compared to the naturally occurring NIF.

Substitution

As used herein “substitution” results from the replacement of one or more nucleotides or amino acids by different nucleotides or amino acids, respectively.

Homologue

The term “homologue” with respect to the nucleotide sequences of NIF shown in SEQ ID NO: 1 or 2 and the amino acid sequence of NIF shown in SEQ ID NO: 3 or 4 may be synonymous with allelic variations of the sequences.

In particular, the term “homology” as used herein may be equated with the term “identity”. Here, sequence homology with respect to the nucleotide sequence of the present invention and the amino acid sequence of the present invention can be determined by a simple “eyeball” comparison (i.e. a strict comparison) of any one or more of the sequences with another sequence to see if that other sequence has at least 70%, preferably at least 80%, more preferably at least 85% homology to the sequence shown in SEQ ID NO: 3 or 4. Preferably there is at least 90%, more preferably at least 95%, most preferably at least 98% homology to the sequence shown in SEQ ID NO: 3 or 4. Relative sequence homology (i.e. sequence identity) can also be determined by commercially available computer programs that can calculate percentage (%) homology between two or more sequences. Typical examples of such computer programs are CLUSTAL or BLAST.

Percentage (%) homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

Combination Therapy

A reduction in neuronal damage following acute ischaemic stroke can be achieved by three major strategies: (1) restoration of cerebral blood flow through the use of thrombolytics/fibrinolytics, such as t-PA, (2) prevention of secondary “reperfusion” injury (anti-neutrophilic action), and (3) inhibition of the pathophysiological cascades that occur as a result of decreased cerebral blood flow through the use of neuroprotective agents. Thrombolytics/fibrinolytics and neuroprotective agents are currently being investigated individually in clinical trials. The only available treatment for acute ischaemic stroke is the thrombolytic, recombinant tissue plasminogen activator (rt-PA), which has been shown to improve clinical outcome if given within 3 hours of the onset of ischaemic stroke (The National Institute of Neurological Disorders and Stroke rt-PA Stroke study group. Tissue plasminogen activator for acute ischaemic stroke. N. Eng. J. Med. 1995; 333; 1581-87). However, only a small fraction of the total number of stroke patients can be treated with rt-PA because most patients reach the hospital long after the 3-hour therapeutic window. One of the detrimental effects of delayed (>3 hours) treatment with rt-PA is that there is an increased likelihood of cerebral haemorrhagic transformation. In addition, reperfusion at points beyond 3 hours may not reduce the volume of cerebral infarction (Zivin J A. Neurology 1998; 50; 599-563). The beneficial effects of rt-PA for the treatment of acute ischaemic stroke is, therefore, limited by the small time window in which the compound may be administered safely.

Treatment of stroke with a combination of therapeutic approaches—thrombolysis and neuroprotectants—might result in a number of added benefits to the stroke patient. For example, thrombolysis-induced reperfusion and neuroprotective agents may act together to give greater benefits than when such mechanisms/agents act in isolation. Thus combining both strategies may result in a more complete attenuation of neuronal damage and a better clinical outcome than from either of the two treatments alone. In addition, if the neuroprotectant drug is administered early, it may prolong the time interval during which the brain can withstand ischaemia prior to reperfusion. Thus, it may extend the time window for thrombolytic therapy. Zhang et al. (Zhang et al., Neurology, 52: 273-279 (1999)) investigated the efficacy of an anti-leukocyte adhesion antibody (anti-CD18) as an adjuvant for delayed (2 hours and 4 hours) thrombolytic therapy (rt-PA) in a rat model of focal embolic stroke. When assessed at either 48 h or 168 h after the ischaemic insult, co-administration of rt-PA and anti-CD18 antibody 2 hours and 4 hours after embolization reduced infarct volume significantly and improved neurologic deficits when compared with a control group or a group treated with rt-PA alone. Thus, the combination of rt-PA and anti-leukocyte adhesion antibody treatments may extend the therapeutic window of thrombolysis.

Hence the present invention, which discloses the combined therapeutic use of at least one NIF and at least one neuroprotective or thrombolytic/fibrinolytic agent and shows that, for example, NIF+recombinant human t-PA (rht-PA) can enhance the efficacy of thrombolytic/fibrinolytic therapy and can extend the therapeutic time window for such use.

Animal studies have shown that administration of Neutrophil Inhibitory Factor (NIF) can reduce ischaemic cell damage and improve neurologic outcomes in a model of transient focal ischaemia (Jiang et al. Ann Neurol 1995;38:935-42; Jiang et al. Brain Res 1998; 788:25-34). NIF is a selective antagonist of the β₂ integrin CD11b/CD18 and thus blocks a number of neutrophil adhesion-dependent functions that are mediated by this integrin receptor. One of the detrimental effects of delayed (>3.0 h) treatment with rt-PA is that reperfusion induced with t-PA increases the likelihood of cerebral haemorrhagic transformation possibly due to endothelial dysfunction. Thus, by reducing the secondary damage resulting from adhesion of neutrophils to the cerebral microvasculature by administering NIF, the therapeutic intervention against stroke using thrombolytic therapy (for example, using t-PA) may be benefited and be augmented.

Combination therapy with neuroprotectants and thrombolytics in acute ischaemic stroke has been reviewed by Thorsten Steiner and Werner Hacke in European Neurology (1998), 40:1-8. However, the use of a NIF in such a combination therapy is not disclosed.

The combination therapy (including uses, methods, pharmaceutical compositions, processes and products) of the present invention thus utilises the therapeutic benefits of two or more compounds, i.e. at least one NIF and at least one other compound which is a neuroprotective or thrombolytic/fibrinolytic agent, preferably t-PA, more preferably recombinant t-PA (rt-PA), most preferably recombinant human t-PA (rht-PA). Said rht-PA is preferably Alteplase (Genentech, San Francisco, Calif., USA), Monteplase (Eisai Co. Ltd., Japan) or Tenecteplase (Genentech).

Combination “Partner Compounds” with NIF

Combination therapy with neuroprotectants and thrombolytics in acute ischaemic stroke has been reviewed by Thorsten Steiner and Werner Hacke in European Neurology (1998), 40:1-8. However, the use of a NIF in such a combination therapy is not disclosed. Nevertheless, this review article discloses a number of suitable combination “partner compounds” for use with NIF in accordance with the present invention (and is therefore incorporated herein by reference). For the most part, the disclosed “partner compounds” in the review article fall within one or more of the following categories of compounds.

“Clot-busting” Agents (e.g. Thrombolytics/fibrinolytics)

“Clot-busting” agents, such as thrombolytic/fibrinolytic agents, are compounds, specifically proteins and (poly)peptides, which restore or improve cerebral blood flow by dissolving the embolus or thrombus that causes the artery occlusion (thrombolysis).

Particularly preferred for use in the present invention along with at least one NIF is at least one thrombolytic/fibrinolytic agent. Examples of preferred suitable thrombolytic/fibrinolytic agents that can be employed in the methods and pharmaceutical compositions of this invention, as described above, are plasminogen activators, such as tissue plasminogen activator (t-PA; and its variants such as, inter alia, Alteplase, Monteplase, Reteplase, Lanoteplase, Duteplase and Tenecteplase) or Desmoteplase, urokinase, pro-urokinase, streptokinase, p-anisoylated plasminogen streptokinase activator complex (APSAC), urokinase plasminogen activator (uPA) and MMP inhibitors.

Plasminogen Activators

Plasminogen activators are a family of proteases which characteristically catalyse the enzymatic conversion of plasminogen to plasmin. Examples of plasminogen activators suitable for use in the present invention are tissue plasminogen activator (t-PA; and its variants such as, inter alia, Alteplase, Monteplase, Reteplase, Lanoteplase, Duteplase and Tenecteplase) or Desmoteplase. Tissue plasminogen activator (t-PA) and Desmoteplase, inter alia, catalyse the enzymatic conversion of plasminogen to plasmin through the hydrolysis of a single Arginine-Valine bond.

Tissue Plasminogen Activator

Tissue plasminogen activator (also known as fibrinokinase, extrinsic plasminogen activator, t-PA or TPA) is a glycoprotein and has an approximate molecular weight (MW) of about 70,000 Daltons (68,000 Daltons). It is a serine protease which catalyses the enzymatic conversion of pro-enzyme plasminogen to active enzyme plasmin through the hydrolysis of a single Arginine-Valine bond. The catalytic site of t-PA is composed of amino acids His-322, Asp-371 and Ser-478. t-PA is a poor plasminogen activator in the absence of fibrin. The amino-terminal region is composed of several domains, which are homologous to other proteins. These distinct domains are involved in several functions of the enzyme, including binding to fibrin, fibrin-specific plasminogen activation, binding to endothelial cell receptors and rapid clearance in vivo. One such domain, comprising amino acid residues 50 to 87 (E domain) is homologous to Human Epidermal Growth Factor and seems to be involved in fibrin binding, fibrin affinity and in vivo clearance. The t-PA cDNA was cloned and subsequently expressed in Chinese hamster ovary (CHO) cells.

Tissue plasminogen activator (t-PA) is a component of the mammalian fibrinolytic system responsible for the specific activation of plasminogen associated with fibrin clots (i.e. it is capable of dissolving blood clots) and is described in detail in U.S. Pat. No. 5,976,530 which issued in Nov. 2, 1999, and which is incorporated herein by reference.

By “tissue plasminogen activator” and “t-PA” (and the like) is meant any polypeptide sequence having tissue plasminogen activator activity, and includes, but is not limited to recombinantly produced t-PA (rt-PA), preferably human recombinant t-PA (rht-PA), e.g. Alteplase (Genentech), most preferably rht-PA variants, such as Reteplase (Boehringer Mannheim, Germany), Monteplase (Eisai Co. Ltd., Japan), Lanoteplase (Genetics Institute, US), Duteplase (Genetics Institute, US; Baxter International, US) or Tenecteplase (Genentech; Tenecteplase is a point mutation of wild-type t-PA—see below).

Alteplase is also known as Actase, Actilyse, Actiplas, Activacin, Activase®, plasminogen activator (human tissue-type protein moiety) or t-PA and is a recombinant single-chain plasminogen activator originated by Genentech. Alteplase is disclosed in EP 0093619 B.

Reteplase is also known as Retavase™, Retevase, Ecokinase, BM 06022, recombinant plasminogen activator (rPA), 173-L-serine-174-L-tyrosine-175-L-glutamine-173-527-plasminogen activator (human tissue-type) or Rapilysin® and is an unglycosylated recombinant tissue plasminogen activator (rPA) consisting of the kringle 2 and protease domains of human t-PA expressed in Escherichia coli cells originated by Boehringer Mannheim. Reteplase is disclosed in EP 0382174 B.

Monteplase (also known as Cleactor®, E 6010, Mf-tPA, tPA or angiokinase) is a modified second-generation t-PA developed and launched by Eisai Co. Ltd. (Japan) as an anti-thrombotic. The agent is a recombinant tissue plasminogen activator that has been modified by 1 amino acid (plasminogen activator (human tissue-type protein moiety reduced) 84-L-serine) in the epidermal growth factor domain compared with Alteplase (Genentech). Specifically, Monteplase was constructed by substituting the amino acid residue Cys-84 for serine in the Epidermal Growth Factor domain (E domain) of native t-PA, by site-directed mutagenesis and was expressed in baby Syrian hamster kidney (BHK) cells. The expressed protein was purified from conditioned medium by affinity chromatography through a column in which monoclonal anti-t-PA antibody was coupled to a gel matrix. The molecular weight (MW) of the recombinant product was approximately 70,000 Daltons, with a specific activity of 150000 UI/mg. The Cys-84 to serine (C84S) mutation results in a t-PA with a longer plasma half-life than Alteplase and so can be administered by injection rather than infusion (single bolus). Its effects on clot lysis are more potent and longer-lasting than those of Alteplase.

Lanoteplase is also known as BMS 200980, FEX 1, Oneplas, SUN 9216, nPA or N-[N2-(N-glycyl-L-alanyl)-L-arginyl]-117-L-glutamine-245-L-methionine-(1-5)-(87-527)-plasminogen activator (human tissue-type protein moiety) and is a novel second-generation tissue plasminogen activator originated by Genetics Institute. It is a plasminogen/plasminogen activator chimera that has the fibrin-binding kringle 1 domain of plasminogen and 2 kringle and the serine protease domain of the wild-type tissue plasminogen activator. Lanoteplase is disclosed in EP 0293394 B.

Duteplase is also known as Prolysis, Tiplagen, SM 9527, 245-L-methionine plasminogen activator (human tissue-type 2-chain form protein moiety) or Solclot and is a double-chain tissue plasminogen activator, closely related to the single-chain Alteplase originated by Genetics Institute/Baxter International using recombinant technology. It is a recombinant variant (valine (V)→methionine (M) substitution at position 245 (=V245M) of Alteplase) of naturally occurring human tissue-type plasminogen activator. Duteplase is disclosed in Yakuri to Chiryo (1996), 24(4), 795-798.

Tenecteplase is also known as TNK, TNK-tPA, TNKase™, 103-L-asparagine-117-L-glutamine-296-L-alanine-297-L-alanine-298-L-alanine-299-L-alanine plasminogen activator (human tissue-type) or Metalyse® and is a second-generation plasminogen activator originated by Genentech. It is a bioengineered variant of Activase®, which is a recombinant DNA-derived variant of naturally-occurring human t-PA. It is constructed with amino acid substitutions at three sites (the letters T, N and K represent the three regions changed from the natural t-PA protein). Specifically, Tenecteplase is a genetically engineered variant of t-PA and is similar to wild-type t-PA, but has amino acid substitutions at 3 sites: a threonine (T) is replaced by asparagine (N), which adds a glycosylation site to position 103; an asparagine (N) is replaced by glutamine (Q), thereby removing a glycosylation site from site 117; and 4 amino acids, lysine (K), histidine (H), and 2 arginines (R), are replaced by 4 alanines (A) at the third site (i.e.=t-PA modified at the T103N, N117Q and KHRR(296-299)AAAA sites; B. A. Keyt, N. F. Paoni, C. J. Refino, L. Berleau, H. Nguyen, A. Chow, A faster-acting and more potent form of tissue plasminogen activator, Proc. Natl. Acad. Sci. USA 91 (1994) 3670-3674). Together, these substitutions have led to: a prolonged half-life that enables single-bolus dosing; an enhanced specificity for fibrin, a key component of intracoronary clots (14-fold greater than wild-type t-PA), which results in less disturbance of the body's coagulation, or natural clotting, system; and an increased level of resistance to type 1 plasminogen activator inhibitor (PAI-1), an inhibitory protein that can interfere with the clot-dissolving effects of a thrombolytic (Keyt et al. (1994)).

Since platelets which contain high levels of PAI-1 have been demonstrated in thromboemboli, Tenecteplase with increased PAI-1 resistance may enhance thrombolysis. Indeed, Tenecteplase produces significantly faster and more complete recanalization of occluded arteries in a rabbit model of carotid artery thrombosis and evokes less systemic activation of plasminogen and haemorrhagic transformation in a rabbit model of cerebral embolic ischaemia compared with wild type t-PA (G. R. Thomas, H. Thibodeaux, C. J. Errett, J. M. Badillo, B. A. Keyt, C. J. Refino, A long-half-life and fibrin-specific form of tissue plasminogen activator in rabbit models of embolic stroke and peripheral bleeding, Stroke 25 (1994) 2072-2078; C. R. Benedict, C. J. Refino, B. A. Keyt, R. Pakala, N. F. Paoni, G. R. Thomas, New variant of human tissue plasminogen activator (TPA) with enhanced efficacy and lower incidence of bleeding compared with recombinant human TPA, Circulation 92 (1995) 3032-3040). Tenecteplase is disclosed in EP 0643772 B.

Genentech's new t-PA variant Tenecteplase represents an improvement over Genentech's first-generation t-PA (Alteplase), in that it can be given over five seconds, rather than a 90-minute infusion, and in one dose.

Provided below is a list of tissue plasminogen activators (wild-type and variants). NIF and any of its variants described above can be used in combination with any of the above-mentioned t-PA types as well as, inter alia, any of the following t-PA types in accordance with the present invention:

Data entry =
ACCESSION NUMBER (GenSeqP and GeneSeqN database entries)
Description of t-PA(Variations over wild-type usually indicated by
“X₁-Residue Position-X₂”, e.g. R275G = Arginine (R)
substituted for Glycine (G) at position 275)
N.B. a Table of amino acids and their symbols can be
found below
APPLICANT (Company)
PATENT (APPLICATION) NUMBER + PRIORITY DATE
(Year-Month-Day)

Amino Acids and their Symbols

| | | | | | |
|---|---|---|---|---|---|
| A | Alanine | (Ala) | M | Methionine | (Met) |
| C | Cysteine | (Cys) | N | Asparagine | (Asn) |
| D | Aspartic Acid | (Asp) | P | Proline | (Pro) |
| E | Glutamic Acid | (Glu) | Q | Glutamine | (Gln) |
| F | Phenylalanine | (Phe) | R | Arginine | (Arg) |
| G | Glycine | (Gly) | S | Serine | (Ser) |
| H | Histidine | (His) | T | Threonine | (Thr) |
| I | Isoleucine | (Ile) | V | Valine | (Val) |
| K | Lysine | (Lys) | W | Tryptophan | (Trp) |
| L | Leucine | (Leu) | Y | Tyrosine | (Tyr) |

AAP30001

Sequence of full-length tissue plasminogen activator (t-Pa)

GENENTECH INC.

EP93619-A. May 5, 1982

AAN50223

cDNA sequence encoding tissue plasminogen activator

CIBA GEIGY AG

EP143081-A. Nov. 21, 1983

AAP50342

Human tPA

GENETICS INST

DK8406107-A. Dec. 27, 1983

AAP60214

Sequence of active human uterine tissue plasminogen activator (UTPA)

INTEG GENETICS INC.

EP178105-A. Oct. 1, 1984

AAW47535

Tissue plasminogen activator variant R275G

GENENTECH INC.

U.S. Pat. No. 5,714,372-A. Apr. 22, 1985

AAP60810

Sequence of modified human tissue plasminogen activator (t-PA)

GENENTECH INC.

FR2581652-A. Apr. 22, 1985

AAW47536

Tissue plasminogen activator variant R275E

GENENTECH INC.

U.S. Pat. No. 5,714,372. Apr. 22, 1985

AAW47537

Tissue plasminogen activator variant 1276P

GENENTECH INC.

AAN70248

Sequence encoding human tissue plasminogen activator (tPA) produced by normal human cells

MITSUI TOATSU CHEM INC.

EP225177-A. Nov. 27, 1985

AAQ86576

Human tissue plasminogen activator cDNA

MITSUI TOATSU CHEM INC.

JP07046983-A. Nov. 27, 1985

AAW23368

Human tissue plasminogen activator deletion mutant

BEHRINGWERKE AG./CHIRON CORP.

U.S. Pat. No. 5,656,269. Dec. 23, 1985

AAP60790

Sequence of human pre-tissue plasminogen activator (pre-t-PA)

GENENTECH INC.

GB2173804-A. Apr. 1, 1986

AAP81913

Tissue plasminogen activator encoded by pEMp1-tPA

DAMON BIOTECH INC.

WO8800242-A. Jun. 26, 1986

AAP93716

Human melanoma t-PA encoded by plasmid pKG12

KABIGEN AB.

EP297066-A. Jun. 18, 1987

AAP94238

Human tissue plasminogen activator (t-PA) gene

GENENTECH INC.

WO8900197-A. Jun. 30, 1987

AAR13441

MB1023 t-PA variant

MONSANTO CO.

U.S. Pat. No. 5,037,752. Oct. 9, 1987

AAR96220

Full-length tissue plasminogen activator

ZYMOGENETICS INC.

U.S. Pat. No. 5,504,001. Nov. 25, 1987

AAP90916

Human tissue plasminogen activator

FUJISAWA PHARM. KK.

JP0174388-A. Dec. 28, 1987

AAR09284

Sequence of tissue plasminogen activator (tPA) mutant Thr 478

UNIV. OF WASHINGTON

WO8912680-A. Jun. 20, 1988

AAR09286

Sequence of tissue plasminogen activator analogue BBNT5 (Ser 67Ser 68)

BRIT BIO-TECHN LTD.

WO8912681-A. Jun. 24, 1988

AAR09289

Sequence of tissue plasminogen activator analogue BBNT12 (Asp 67Thr 68)

BRIT BIO-TECHN LTD.

WO8912681-A. Jun. 24, 1988

AAR09288

Sequence of tissue plasminogen activator analogue BBNT11 (Ser 67Leu 68)

BRIT BIO-TECHN LTD.

WO8912681-A. Jun. 24, 1988

AAR04699

Native tissue plasminogen activator (t-PA)

NOVO-NORDISK A/S

EP351246-A. Jul. 15, 1988

AAR04702

Sequence of tissue plasminogen activator (t-PA) analogue t-PA

C87S;K419S with altered residues 87 and 419

NOVO-NORDISK A/S

EP351246-A. Jul. 15, 1988

AAR04700

Sequence of tissue plasminogen activator (t-PA) analogue t-PA

C87SH420S with altered residues 419 and 420

NOVO-NORDISK A/S

EP351246-A. Jul. 15, 1988

AAR04701

Sequence of tissue plasminogen activator (t-PA) analogue t-PA K419S with altered residue 419

NOVO-NORDISK A/S

EP351246-A. Jul. 15, 1988

AAQ05177

Sequence encoding thrombolytic protein with secondary structure of human tissue plasminogen activator

YAMANOUCHI PHARM KK.

JPO2145184-A. Nov. 29, 1988

AAR13727

T-PA67+mutant with supernumerary N-linked oligosaccharide side chain

UNIV. OF TEXAS SYST. (COLD-) COLD SPRING HARBOR LAB.

U.S. Pat. No. 5,041,376. Dec. 9, 1988

AAQ04903

Part of tPA024 gene encoding precursor protein

YAMANOUCHI PHARM KK.

EP373896-A. Dec. 12, 1988

AAQ04904

Part of tPA023 gene encoding precursor protein

YAMANOUCHI PHARM KK

EP373896-A. Dec. 12, 1988

AAR06237

Novel tissue plasminogen activator (tPA) encoded by plasmid pST112

FUJISAWA PHARM KK.

EP379890-A. Jan. 23, 1989

AAQ11551

Sequence encoding tissue plasminogen activator derivative

KANEGAFUCHI CHEM KK.

JP03065184-A. Aug. 3, 1989

AAR12847

T-PA Kringle 1 domain substitution mutant

KANEGAFUCHI CHEM KK.

JP03127987-A. Oct. 11, 1989

AAR23801

Zymogen-like t-PA (His 305)

UNIV. TEXAS SYSTEM

WO9206203-A. Sep. 28, 1990

AAR23807

t-PA (Tyr 297) mutant

UNIV. TEXAS SYSTEM

WO9206203-A. Sep. 28, 1990

AAR23811

t-PA (Glu 296 Glu 298 Glu 299) triple mutant

UNIV. TEXAS SYSTEM

WO9206203-A. Sep. 28, 1990

AAR23810

t-PA (Gly 301) mutant

UNIV. TEXAS SYSTEM

WO9206203-A. Sep. 28, 1990

AAR23809

t-PA (Glu 299) mutant

UNIV. TEXAS SYSTEM

WO9206203-A. Sep. 28, 1990

AAR23808

t-PA (Glu 298) mutant

UNIV. TEXAS SYSTEM

WO9206203-A. Sep. 28, 1990

AAR23806

t-PA (Glu 296) mutant

UNIV. TEXAS SYSTEM

WO9206203-A. Sep. 28, 1990

AAR23802

Zymogen-like t-PA (Ser 292 His 305)

UNIV. TEXAS SYSTEM

WO9206203-A. Sep. 28, 1990

AAR23804

t-PA (Glu 304) mutant

UNIV. TEXAS SYSTEM

WO9206203-A. Sep. 28, 1990

AAR23803

t-PA (Ser 304) mutant

UNIV. TEXAS SYSTEM

WO9206203-A. Sep. 28, 1990

AAR44834

Human tPA (R129W)

TAKEDA CHEM IND LTD.

JP05304992-A. Jun. 20, 1991

AAR38674

Sequence of tissue plasminogen activator (t-PA)

GENENTECH INC.

WO9312225-A. Dec. 16, 1991

AAV37294

Human tissue plasminogen activator gene sequence

HARVARD COLLEGE

U.S. Pat No. 5,780,272. Sep. 10, 1993

AAY43397

Human tissue plasminogen activator protein sequence

CANGENE CORP.

U.S. Pat. No. 5,985,607. Dec. 19, 1994

AAY50868

Human tissue plasminogen activator protein fragment

OKLAHOMA MEDICAL RES FOUND.

WO9957251 -A2. May 6, 1998

AAY99590

Human tissue-type plasminogen activator t-PA

OKLAHOMA MEDICAL RES FOUND.

WO0032759-A1. Dec. 2, 1998

AAQ53318

Human tPA (R129W) coding sequence

TAKEDA CHEM IND LTD.

JP05304992-A. Nov. 19, 1993

BC007231

Homo sapiens, plasminogen activator, tissue, clone Mammalian Gene Collection (MGC): 15287 . . . difference from wild-type t-PA=nucleic acid change at position 501 t→c

NIH-MGC Project URL: http://mgc.nci.nih.gov

Genzyme Corporation (US) was developing (as well as Integrated Genetics, Inc. and Toyobo Co. Ltd.) a recombinant tissue-type plasminogen activator (human uterine tissue plasminogen activator), known as plasminogen activator-2, tPA-2 or LatPA (EP 0178105A), which is identical to human t-PA (Alteplase) produced by Genentech.

Substantial advantages can be achieved by making changes in the wild-type t-PA amino acid sequence. Not only can activity be increased but at the same time sensitivity to plasminogen activator inhibitor can be decreased, so that an overall a very substantial increase in effective activity can be achieved in vivo. Also, the enzyme can be made substantially more specific in providing for enhanced fibrin dependence, so that it has substantially reduced activity in the absence of clots.

Accordingly, Hoechst AG (Germany) was developing a recombinant tissue-type plasminogen activator known as plasminogen activator-2. Also Hoechst Marion Roussel Deutschland GmbH (Germany) and Chiron Corporation (Emeryville, Calif., USA) describe a human tissue plasminogen activator having lysine 277 substituted with another amino acid and further comprises a deletion of 3-25 amino acids from the C-terminus (see U.S. Pat. No. 5,976,530 issued Nov. 2, 1999).

British Biotechnology plc (UK) is developing a recombinant protein plasminogen activator-2 (also known as tPA-2 or BBNT-12 and subject of patent application WO 95/35117).

Novartis AG (Basle, Switzerland) was developing CGP-42935 (also known as plasminogen activator, K2tuPA or angiokinase), a 173-275-plasminogen activator [173-serine, 174-tyrosine, 175-glutamine] (human tissue-type reduced) fusion protein with urokinase (human urine B-chain reduced). It is hybrid plasminogen activator linking the kringle 2 domain of t-PA to the catalytic protease domain of scu-PS.

Menarini Richerche Sud SpA is developing Amediplase (also known as plasminogen activator, K2tuPA or MEN 9036), a chimeric molecule containing functional domains of both t-PA (kringle 2 domain from the t-PA A-chain) and pro-urokinase (carboxy terminal region), and a pegylated variant of staphylokinase with reduced immunogenicity. This second generation t-PA has a reduced plasma clearance and can be administered in a single bolus. Amediplase is identical to CGP-42935 (see above) and resulted from a collaborative project with Novartis AG. Amediplase is described in EP 0277313 and Nature Biotechnology (1997), 15, 405.

Mitsui Pharmaceuticals (now Nihon Schering) (Japan) originated Nateplase (also known as Milyzer®, Tepase®, MMR-701, plasminogen activator or t-PA), a recombinant single-chain human tissue-type plasminogen activator, which is described in EP 0225177A (in the name of Mitsui Toatsu Chem. Inc.).

The biochemistry and pharmacology of some t-PA variants produced by mutagenesis is described in Annual Review of Pharmacology and Toxicology (1990), Volume 30, pages 91-121 (which is incorporated herein by reference). The t-PA variants described in this publication can be readily used in a combination therapy with NIF, including uses, methods, pharmaceutical compositions, processes and products in accordance with the present invention.

Yet another t-PA variant that can be used in the present invention is Tisokinase (see Yakuri to Chiryo (1996), 24(4), 795-798).

Desmoteplase

Desmoteplase (Accession Number P49150; reference: Gene 105(2):229-237 (1991)) is also known as plasminogen activator (Desmodus rotundus isoform α1 protein moiety reduced) and is from the plasminogen activator family cloned from the salivary gland of the vampire bat Desmodus rotundus (=vampire bat venom) and originated from Schering A G, Berlin, Germany (developed by Paion, Germany). It belongs to peptidase family S1 (also known as the trypsin family) and contains 1 kringle domain and specifically cleaves the Arginine-Valine bond in plasminogen to form plasmin. Its predicted amino acid sequences display structural features also found in tissue-type plasminogen activator. The largest forms (DSPAα1 and DSPAα2) contain a signal peptide, a finger (F), an epidermal growth factor (EGF), a kringle, and a serine protease domain, whereas DSPAβ and DSPAγ lack the F and F-EGF domains, respectively.

Merck & Co., Inc. was developing Desmoteplase known as “(vampire bat) plasminogen activator”, bat-tPA or angiokinase (see EP 0352119A and Accession Numbers AAQ00543 and AAR05122). This vampire bat glycosylated plasminogen activating protein, which needs fibrin co-factor to activate plasminogen, has greater selectivity for fibrin-bound plasminogen than t-PA.

Further Variants of Wild-type Tissue Plasminogen Activator

Provided below is a list of further variants of wild-type tissue plasminogen activator. NIF and any of its variants described above can be used in combination with any of the above-mentioned t-PA types as well as, inter alia, any of the following t-PA types in accordance with the present invention:

Data entry =
ACCESSION NUMBER (GenSeqP and GeneSeqN database entries)
Description
Reference(s)

BC002795

Homo sapiens, similar to plasminogen activator, tissue, clone MGC: 3677

IMAGE: 3618149, mRNA, complete cds

NIH-MGC Project URL: http://mgc.nci.nih.gov

X13097

Human mRNA for tissue type plasminogen activator

Nucleic Acids Research 18(4), 1086 (1990)

AF260825

Homo sapiens neonatal thrombolytic agent alpha-form mRNA, partial cds

X02901

Human mRNA for finger-domain lacking tissue-type plasminogen (t-PA)

Nature 301 (5897), 214-221 (1983)

Proc. Natl. Acad. Sci. USA 80(2), 349-352 (1983)

FEBS Lett. 189(1), 145-149 (1985)

NP_{—}000921

Plasminogen activator, tissue-type isoform 1 preproprotein

Nature 301 (5897), 214-221 (1983)

Proc. Natl. Acad. Sci. USA 81(17), 5355-5359 (1984)

Gene 42(1), 59-67 (1986)

J. Biol. Chem. 261(15), 6972-6985 (1986)

Mol. Biol. Med. 3(3), 279-292 (1986)

Gene 63(2), 155-163 (1988)

Nucleic Acids Research 16(12), 5695 (1988)

Nucleic Acids Research 18(4), 1086 (1990)

Agric. Biol. Chem. 55(5), 1225-1232 (1991)

J. Mol. Biol. 258(1), 117-135 (1996)

NP_{—}000922

Plasminogen activator, tissue-type isoform 2 precursor

As for NP_{—}000921 above

M26666

Synthetic human tissue-type plasminogen activator mRNA, complete cds

Gene 63(2), 155-163 (1988)

Neuroprotective Agents

Neuroprotective agents are compounds which have an affect on the biochemical and metabolic consequences of ischaemic brain injury in order to prevent neuronal cell death in the penumbra (neuroprotection).

Also preferred for use in the present invention along with at least one NIF is at least one NMDA receptor antagonist, preferably an NMDA glycine site antagonising compound or a pharmaceutically acceptable salt thereof. Examples of NMDA glycine site antagonists that are suitable for use in the pharmaceutical compositions and methods of this invention are those referred to in the following: U.S. Pat. No. 5,942,540, which issued on Aug. 24, 1999; PCT Application WO 99/34790, which was published on Jul. 15, 1999; WO 98/47878, which was published on Oct. 29, 1998; PCT Application WO 98/42673, which was published on Oct. 1, 1998; European Patent Application EP 0966475 A1, which was published on Dec. 29, 1991; PCT Application WO 98/39327, which was published on Sep. 11, 1998; PCT Application WO 98/04556, which was published on Feb. 5, 1998; PCT Application WO 97/37652, which was published on Oct. 16, 1997; PCT Application WO 97/32873, which was published on Sep. 12, 1997; PCT Application WO 98/38186, which was published on Sep. 3, 1998; U.S. Pat. No. 5,837,705, which was issued on Oct. 9, 1996; PCT Application WO 97/20553, which was published on Jun. 12, 1997; U.S. Pat. No. 5,886,018, which was issued on Mar. 23, 1999; U.S. Pat. No. 5,801,183, which was issued on Sep. 1, 1998; PCT Application WO 95/07887, which was published on Mar. 23, 1995; U.S. Pat. No. 5,686,461, which was issued on Nov. 11, 1997; U.S. Pat. No. 5,614,509, which was issued on Mar. 25, 1997; U.S. Pat. No. 5,510,367, which was issued on Apr. 23, 1996; European Patent Application 0517347 A1, which was published on Dec. 9, 1992; and U.S. Pat. No. 5,260,324, which published on Nov. 9, 1993.

Other examples of NMDA glycine site receptor antagonists that can be used in the pharmaceutical composition and methods of this invention are N-(6,7-dichloro-2,3-dioxo-1,2,3,4-tetrahydro-quinoxalin-5-yl)-N-(2-hydroxy-ethyl)-methanesulfonamide, 6,7-dichloro-5-[3-methoxymethyl-5-(1-oxy-pyridin-3-yl)-[1,2,4]triazol-4-yl]-1,4-dihydro-quinoxa-line-2,3-dione, and (−)-6,7-dichloro-5-[3-methoxymethyl-5-(1-oxidopyridin-3-yl)-4H-1,2,4-triazol-4-yl]-2,3(1H,4H)-quinoxalinedione. Another example of a NMDA glycine site receptor antagonist is GV150526 (GlaxoSmithKline, UK).

Also preferred for use in the present invention along with at least one NIF is at least one AMPA/kainate receptor antagonising compound or a pharmaceutically acceptable salt thereof. Examples of suitable AMPA/kainate receptor antagonising compounds that can be employed in the methods and pharmaceutical compositions of this invention, as described above, are 6-cyano-7-nitroquinoxalin-2,3-dione (CNQX), 6-nitro-7-sulphamoylbenzo[f]quinoxaline-2,3-dione (NBQX), 6,7-dinitroquinoxaline-2,3-dione (DNQX), 1-(4-aminophenyl)-4-methyl-7,8-methylenedioxy-5H-2,3-benzodiazepine hydrochloride and 2,3-dihydroxy-6-nitro-7-sulfamoylbenzo-[f]quinoxaline.

Also preferred for use in the present invention along with at least one NIF is at least one sodium channel blocking compound or a pharmaceutically acceptable salt thereof. Examples of suitable sodium channel blocking compounds (i.e. sodium channel antagonists) that can be employed in the methods and pharmaceutical compositions of this invention, as described above, are ajmaline, procainamide, flecainide and riluzole.

Also preferred for use in the present invention along with at least one NIF is at least one calcium channel blocking compound or a pharmaceutically acceptable salt thereof. Examples of suitable calcium channel blocking compounds (i.e. calcium channel antagonists) that can be employed in the methods and pharmaceutical compositions of this invention, as described above, are diltiazem, omega-conotoxin GVIA, methoxyverapamil, amlodipine, felodipine, lacidipine, mibefradil, nimodipine and lifarizine.

Also preferred for use in the present invention along with at least one NIF is at least one potassium channel opening compound or a pharmaceutically acceptable salt thereof. Examples of suitable potassium channel openers that can be employed in the methods and pharmaceutical compositions of this invention, as described above, are diazoxide, flupirtine, pinacidil, levcromakalim, rilmakalim, chromakalim, PCO-400 (J. Vasc. Res., November-December 1999, 36 (6), 516-23), SKP-450 (2-[2″(1″, 3″-dioxolone)-2-methyl]-4-(2′-oxo-1′-pyrrolidinyl)-6-nitro-2H-1-benzopyran) and Bristol Myers Squibb's (US) compound BMS 204,352 (Maxi-KCO).

Also preferred for use in the present invention along with at least one NIF is at least one GABA_{A} receptor modulator (e.g. a GABA_{A} receptor agonist) or a pharmaceutically acceptable salt thereof. An example of a suitable GABA_{A} receptor modulator that can be employed in the methods and pharmaceutical compositions of this invention, as described above, is clomethiazole (AstraZeneca, UK).

Other examples of GABA_{A} modulators that can be used in the pharmaceutical compositions and methods of this invention are those that are referred to in the following: PCT Application WO 99/25353, which was published on May 27, 1999; PCT Application WO 96/25948, which was published on Aug. 29, 1996; PCT Application WO 99/37303, which was published on Jul. 29, 1999; U.S. Pat. No. 5,925,770, which was issued on Jul. 20, 1999; U.S. Pat. No. 5,216,159, which was issued on Jun. 1, 1993; U.S. Pat. No. 5,130,430, which was issued on Jul. 14, 1992; U.S. Pat. No. 5,925,770, which was issued on Jul. 20, 1999; and PCT Application WO 99/10347, which was published on Mar. 4, 1999.

Also preferred for use in the present invention along with at least one NIF is at least one NOS inhibiting compound or a pharmaceutically acceptable salt thereof.

There are three known isoforms of NOS—an inducible form (I-NOS) and two constitutive forms referred to as, respectively, neuronal NOS (N-NOS) and endothelial NOS (E-NOS). Each of these enzymes carries out the conversion of arginine to citrulline while producing a molecule of nitric oxide (NO) in response to various stimuli. It is believed that excess nitric oxide (NO) production by NOS plays a role in the pathology of a number of disorders and conditions in mammals. For example, NO produced by I-NOS is thought to play a role in diseases that involve systemic hypotension such as toxic shock and therapy with certain cytokines. It has been shown that cancer patients treated with cytokines such as interleukin 1 (IL-1), interleukin 2 (IL-2) or tumour necrosis factor (TNF) suffer cytokine-induced shock and hypotension due to NO produced from macrophages, i.e. inducible NOS (I-NOS) (see Chemical & Engineering News, Dec. 20, p. 33, (1993)). I-NOS inhibitors can reverse this. It is also believed that I-NOS plays a role in the pathology of diseases of the central nervous system such as ischaemia. For example, inhibition of I-NOS has been shown to ameliorate cerebral ischaemic damage in rats (see Am. J. Physiol., 268, p. R286 (1995)). Suppression of adjuvant induced arthritis by selective inhibition of I-NOS is reported in Eur. J. Pharmacol., 273, p.15-24 (1995).

NO produced by N-NOS is thought to play a role in diseases such as cerebral ischaemia, pain, and opiate tolerance. For example, inhibition of N-NOS decreases infarct volume after proximal middle cerebral artery occlusion in the rat (see J. Cerebr. Blood Flow Metab., 14, p. 924-929 (1994)). N-NOS inhibition has also been shown to be effective in antinociception, as evidenced by activity in the late phase of the formalin-induced hindpaw licking and acetic acid-induced abdominal constriction assays (see Br. J. Pharmacol., 110, p. 219-224 (1993)). In addition, subcutaneous injection of Freund's adjuvant in the rat induces an increase in NOS-positive neurons in the spinal cord that is manifested in increased sensitivity to pain, which can be treated with NOS inhibitors (see Japanese Journal of Pharmacology, 75, p. 327-335 (1997)). Also, opioid withdrawal in rodents has been reported to be reduced by N-NOS inhibition (see Neuropsychopharmacology, 13, p.269-293 (1995)).

Examples of NOS inhibiting compounds that can be used in the methods and pharmaceutical compositions of the present invention are those referred to in: U.S. Provisional Application No. 60/057094, which was filed Aug. 27, 1997 and is entitled “2-Aminopyridines Containing Fused Ring Substituents”; the PCT Application having the same title that was filed on May 5, 1998, which designates the United States and claims priority from Provisional Application No. 60/057094; PCT Application WO 97/36871, which designates the United States and was published on Oct. 9, 1997; U.S. Provisional Patent Application No. 60/057739, entitled “6-Phenylpyridin-2-yl-amine Derivatives”, which was filed on Aug. 28, 1997; PCT Application PCT/IB98/00112, entitled “4-Amino-6-(2-substituted-4-phenoxy)-substituted-pyridines”, which designates the United States and was filed on Jan. 29, 1998; PCT Application PCT/IB97/01446, entitled “6-Phenylpyridyl-2-amine Derivatives”, which designates the United States and was filed on Nov. 17, 1997; and the U.S. Provisional Application, that was filed on Jun. 3, 1998 and is entitled “2-Aminopyridines Containing Fused Ring Substituents”. A further example of a NOS pathway modulator is Lubeluzole.

Also preferred for use in the present invention along with at least one NIF is at least one antioxidant compound or a pharmaceutically acceptable salt thereof. Examples of suitable antioxidant compounds that can be employed in the methods and pharmaceutical compositions of this invention, as described above, are vitamin E (alpha-tocopherol), vitamin A, calcium dobesilate, stobadine, ascorbic acid, alpha-lipoic acid, corcumin, catalase, prevastatin, N-acetylcysteine, nordihydroguaiaretic acid, pyrrolidine dithiocarbamate, LY341122, and Metexyl (4-methoxy-2,2,6,6-tetramethylpiperidine-1-oxyl).

Also preferred for use in the present invention along with at least one NIF is at least one free-radical scavenger or a pharmaceutically acceptable salt thereof. Examples of suitable free-radical scavenger compounds that can be employed in the methods and pharmaceutical compositions of this invention, as described above, are Tirilizid and Ebselen. Also potentially useful is the AstraZeneca (UK)/Centaur (USA) compound NXY-059.

Anti-inflammatory Agents

Also preferred for use in the present invention along with at least one NIF is at least one anti-inflammatory compound or a pharmaceutically acceptable salt thereof. Examples of suitable anti-inflammatory compounds that can be employed in the methods and pharmaceutical compositions of this invention, as described above, are non-steroidal anti-inflammatory drugs (NSAIDs), COX2 inhibitors, dipyridamole, acetaminophen and steroidal anti-inflammatory agents such as methyl prednisolone and cortisone. Examples of NSAIDs are aspirin, diclofenac sodium, nabumetone, naproxen, naproxen sodium, ketorolac, ibuprofen and indomethacin.

Examples of suitable COX2 inhibitors that can be employed in the methods and pharmaceutical compositions of this invention are those referred to in the following: U.S. Provisional Patent Application No. 60/134,311, which was filed on May 14, 1999; U.S. Provisional Patent Application No. 60/134,312, which was filed on May 14, 1999; U.S. Provisional Patent Application No. 60/134,309, which was filed on May 14,1999.

Other examples of suitable COX2 inhibitors that can be employed in the methods and pharmaceutical compositions of this invention are those referred to in the following: U.S. Pat. No. 5,817,700, issued Oct. 6, 1998; PCT Application WO97/28121, published Aug. 7, 1997; U.S. Pat. No. 5,767,291, issued Jun. 16, 1998; U.S. Pat. No. 5,436,265, issued Jul. 25 1995; U.S. Pat. No. 5,474,995, issued Dec. 12, 1995; U.S. Pat. No. 5,536,752, issued Jul. 16, 1996; U.S. Pat. No. 5,550,142, issued Aug. 27, 1996; U.S. Pat. No. 5,604,260, issued Feb. 18, 1997; U.S. Pat. No. 5,698,584, issued Dec. 16, 1997; U.S. Pat. No. 5,710,140, issued Jan. 20, 1998; U.S. Pat. No. 5,840,746, issued Nov. 24, 1998; Great Britain Patent Application 986430, filed Mar. 25, 1998; PCT Application WO97/28120, published Aug. 7, 1997; Great Britain Patent Application 9800689, filed Jan. 14, 1998; Great Britain Patent Application 9800688, filed Jan. 14, 1998; PCT Application WO94/14977, published Jul. 7, 1994; PCT Application WO98/43966, published Oct. 8, 1998; PCT Application WO98/03484, published Jan. 29, 1998; PCT Application WO98/41516, published Sep. 24, 1998; PCT Application WO98/41511, published Sep. 24, 1998; Great Britain Patent Application 2,319,032, issued May 13, 1998; PCT Application WO96/37467, published Nov. 28, 1996; PCT Application WO96/37469, published Nov. 28, 1996; PCT Application WO96/36623, published Nov. 21, 1996; PCT Application WO98/00416, published Jan. 8, 1998; PCT Application WO97/44027, published Nov. 27, 1997; PCT Application WO97/44028, published Nov. 27, 1997; PCT Application WO96/23786, published Aug. 8, 1996; PCT Application WO97/40012, published Oct. 30, 1997; PCT Application WO96/19469, published Jun. 27, 1996; PCT Application WO97/36863, published Oct. 9, 1997; PCT Application WO97/14691, published Apr. 24, 1997; PCT Application WO97/11701, published Apr. 3, 1997; PCT Application WO96/13483, published May 9, 1996; PCT Application WO96/37468, published Nov. 28, 1996; PCT Application WO96/06840, published Mar. 7, 1996; PCT Application WO94/26731, published Nov. 24, 1994; PCT Application WO94/20480, published Sep. 15, 1994; U.S. Pat. No. 5,006,549, issued Apr. 9, 1991; U.S. Pat. No. 4,800,211, issued Jan. 24, 1989; U.S. Pat. No. 4,782,080, issued Nov. 1, 1988; U.S. Pat. No. 4,720,503, issued Jan. 19, 1988; U.S. Pat. No. 4,760,086, issued Jul. 26, 1988; U.S. Pat. No. 5,068,248, issued Nov. 26, 1991; U.S. Pat. No. 5,859,257, issued Jan. 12, 1999; PCT Application WO98/47509, published Oct. 29, 1998; PCT Application WO98/47890, published Oct. 29, 1998; PCT Application WO98/43648, published Oct. 8, 1998; PCT Application WO98/25896, published Jun. 18, 1998; PCT Application WO98/22101, published May 28, 1998; PCT Application WO98/16227, published Apr. 23, 1998; PCT Application WO98/06708, published Feb. 19, 1998; PCT Application WO97/38986, published Oct. 23, 1997; U.S. Pat. No. 5,663,180, issued Sep. 2, 1997; PCT Application WO97/29776, published Aug. 21, 1997; PCT Application WO97/29775, published Aug. 21, 1997; PCT Application WO97/29774, published Aug. 21, 1997; PCT Application WO97/27181, published Jul. 31, 1997; PCT Application WO95/11883, published May 4, 1995; PCT Application WO97/14679, published Apr. 24, 1997; PCT Application WO97/11704, published Apr. 3, 1997; PCT Application WO96/41645, published Dec. 27, 1996; PCT Application WO96/41626, published Dec. 27, 1996; PCT Application WO96/41625, published Dec. 27, 1996; PCT Application WO96/38442, published Dec. 5, 1996; PCT Application WO96/38418, published Dec. 5, 1996; PCT Application WO96/36617, published Nov. 21, 1996; PCT Application WO96/24585, published Aug. 15, 1996; PCT Application WO96/24584, published Aug. 15, 1996; PCT Application WO96/16934, published Jun. 6, 1996; PCT Application WO96/03385, published Feb. 8, 1996; PCT Application WO96/12703, published May 2, 1996; PCT Application WO96/09304, published Mar. 28, 1996; PCT Application WO96/09293, published Mar. 28, 1996; PCT Application WO96/03392, published Feb. 8, 1996; PCT Application WO96/03388, published Feb. 8, 1996; PCT Application WO96/03387, published Feb. 8, 1996; PCT Application WO96/02515, published Feb. 1, 1996; PCT Application WO96/02486, published Feb. 1, 1996; U.S. Pat. No. 5,476,944, issued Dec. 19, 1995; PCT Application WO95/30652, published Nov. 16, 1995; U.S. Pat. No. 5,451,604, published Sep. 19, 1995; PCT Application WO95/21817, published Aug. 17, 1995; PCT Application WO95/21197, published Aug. 10, 1995; PCT Application WO95/15315, published Jun. 8, 1995; U.S. Pat. No. 5,504,215, issued Apr. 2, 1996; U.S. Pat. No. 5,508,426, issued Apr. 16, 1996; U.S. Pat. No. 5,516,907, issued May 14, 1996; U.S. Pat. No. 5,521,207, issued May 28, 1998; U.S. Pat. No. 5,753,688, issued May 19, 1998; U.S. Pat. No. 5,760,068, issued Jun. 2, 1998; U.S. Pat. No. 5,420,343, issued May 30, 1995; PCT Application WO95/30656, published Nov. 16, 1995; U.S. Pat. No. 5,393,790, issued Feb. 28, 1995; and PCT Application WO94/27980, published Feb. 8, 1994.

Also preferred for use in the present invention along with at least one NIF is at least one adenosine A2a receptor agonist. Examples of such adenosine A2a receptor agonists are purine derivatives, preferably adenine derivatives, more preferably 2-aminocarbonyl-9H-purine derivatives. Examples of adenosine A2a receptor agonists can be found in PCT Application WO 00/23457, published Apr. 27, 2000; PCT Applications PCT/IB00/00789, PCT/IB00/01444, PCT/IB00/01446 and PCT/IB01/00167.

Also preferred for use in the present invention along with at least one NIF is at least one NOS inhibiting compound or a pharmaceutically acceptable salt thereof. NOS inhibitors are described above.

Miscellaneous

Also preferred for use in the present invention along with at least one NIF is at least one selective serotonin reuptake inhibitor (SSRI). Examples of SSRIs that can be employed in the methods and pharmaceutical compositions of this invention, as described above, include: fluoxetine, fluvoxamine, paroxetine and sertraline, and pharmaceutically acceptable salts thereof.

Also preferred for use in the present invention along with at least one NIF is at least one anti-CD11/CD18 antibody, preferably monoclonal antibody. An example of such an antibody is cos's (USA) Hu23F2G monoclonal antibody.

Monoclonal antibodies that recognise the counter-receptor of CD11a/CD18 and CD11b/CD18, i.e. ICAM-1 may also be useful in the present invention along with at least one NIF. An example of such an antibody is Enlimomab (Boehringer Ingleheim, Germany).

Also preferred for use in the present invention along with at least one NIF is at least one 5-HT_{1A} agonist, such as Bayer's (Germany) Bay x3702.

Also preferred for use in the present invention along with at least one NIF is at least one immunosuppressant, one β-2 agonist, one antibiotic, or one anti-platelet agent.

In addition to the aforementioned “partner compounds” for use in the present invention, the following broad categories are also contemplated. These “partner compounds” include, but are not limited to: anti-platelet drugs (e.g., Alboaggregin A, BB-2113, BN-50726, BN-50739, ‘Corsevin M’, C68-22, Integrelin, KB-3022, Linotroban, Platelet factor 4, Staurosporine, S-1452, Ticlopidine, TP-9201 and the like), anti-coagulants (e.g., Alpha-1 anti-trypsin, Antithrombin III, Antithrombin polypeptides, Argatroban, Coagulation factor Xa, CTC-110, CTC-111 and other protein C products, CX-397, Dalteparin, Danaproid sodium, Enoxaparin, Factor XIIa inhibitor, Fraxiparine, Heparin, Hirudin, Hirugen, Hoe-023, HV-1, ITF-300 and ITF-1300, Monoclonal antibodies, ONO-3307, Oversulfated LMW heparin, Raviparin sodium, rTAP, R-020, SC-597, Thrombomodulin, TMD1-105 and the like), thrombolytic and related agents (e.g., Kabi-2161, Kunitz protease inhibitor, plasminogen activator inhibitor and the like), anti-ischaemic agents and “neuroprotectives” (e.g., inhibitors of the actions of excitatory amino acids, ACEA-1021, ACPC, Aptiganel, BW-619C, CNS-1145, CNS-1505, CPC-71 and CPC-702, Dextrorphan and dextromethorphan, Eliprodil, ES-242-1, FPL-15896, FR-115427, GP-1-4688, L-687414, L-689560, L-695902, LY-104658, LY-235959, LY-274614, LY-293558, Memantine, NNC-07-9202, NS-257, NPC 17742, “Protara”, Remacemide, Riluzole, SDZ EAA 494, Selfotel, SYM-1010, SYM-1207, YM-90K, MK-801 and the like).

Yet other therapeutic agents useful in the present invention along with at least one NIF are calcium channel blockers (e.g., AJ-394, AK-275, Calpain inhibitors, CD-349, Clentiaze, CNS-1237, CNS-2103, CPC-304 and CPC-317, Dazodipine, Diperdinine, Emopamil, Fasudil, Lacidipine, Lifarizine, Lomerizine, Magnesium, MDL:28170, NB-818, Nilvadipine, Nimodipine, NS-626 and related compounds, SM-6586, SNX-111, S-312-d, U-92032, UK-74505, US-035 and the like), agents targeted at nitric oxide, agents targeted at various other neurotransmitters (e.g., alpha₂-receptor therapeutics, CV-5197, Dopamine receptors, Enadoline, Lazabemide, Milnacipran, Nalmefene, RP-60180, SR-57746A, Synaptic uptake blockers and the like), cytokines, hormones and related products (e.g., AN-100225 and AN-100226, Brain-derived neurotrophic factor, Calcitonin gene-related peptides, CEP-075 and related compounds, Ciliary neurotrophic factor, Endothelial cell factor, Endothelin inhibitors, FR-139317 lnterleukin-1 receptor antagonist (lipocortin), JTP-2942, Macrophage-regulating compounds, Motoneurone trophic factor NBI-117, Nerve growth factor, Neural stem cells, NS-506, NT-3, Posatirelin, Schwann cell promoters, sCR1, Somatomedin-1 and the like), free radical scavengers (e.g., EPC-K1, MCI-186, Nicaraven, Phenazoviridin, Resorstatin, Rumbrin, Superoxide dismutase, Tirilazad mesylate, U-88999E, Yissum project P-0619, YM-737 and the like), gangliosides and related products (e.g., LIGA4, Monosialoganglioside (GM1) ND-37, Siagoside and the like).

Still other classes of therapeutic agents useful in combination with at least one NIF include, but are not limited to: modulators of various specific enzymes (e.g., CEP-217, CEP-245, CEP-392, CNS-1531, Ebselen, Epalrestat, JTP-4819, K-7259, Protease nexin-1, SK-827, Tyrosine kinase modulators, Z-321 and the like), memory enhancers or “nootropics” (e.g., Aloracetam, Choline-L-alfoscerate, DN-2574, Idebenone, Oxiracetam, Piracetam, Pramiracetam, Tacrine and its analogues, Vinconate), neuroprotectives with “diverse” actions (e.g., Ademetionine sulphate tosilate, Ancrod, Apocuanzine, CPC-111, CPC-211, HSV vectors, KF-17329 and KF-19863, LY-178002, MS-153, Nicorandil, N-3393 and N-3398, SUN 4757, TJ-8007, VA-045 and the like), haemorheological agents and blood substitutes (e.g., Drotaverine acephylinate, ‘RheothRx’ Blood substitute and the like) and imaging or contrast agents.

Dosages, Formulations and Administration

This invention relates, inter alia, both to methods of treatment in which at least one NIF and the other active ingredient(s) in the claimed combinations are administered together, as part of the same pharmaceutical composition, as well as to methods in which the two or more active agents are administered separately, as part of an appropriate dose regimen designed to obtain the benefits of the combination therapy. The appropriate dose regimen, the amount of each dose administered, and the intervals between doses of the active agents will depend upon the particular variant of NIF (e.g. rNIF) and the other active ingredient(s) being used in combination, the type of pharmaceutical formulation being used, the characteristics of the subject being treated and the severity of the disorder being treated.

The pharmaceutical combinations may be formulated and used either in combination form (i.e. wherein all the active ingredients are combined into one formulation) or in individual form (i.e. wherein the active ingredients are not combined (or not all combined) into one formulation) as tablets, capsules or elixirs for oral administration; suppositories for rectal administration; sterile solutions, suspensions for injectable administration; and the like. The dose and method of administration can be tailored to achieve optimal efficacy but will depend on such factors as patient weight, diet, concurrent medication and other factors which those skilled in the medical arts will recognise.

Generally, and with respect to the NIF component of the combination therapy (including uses, methods, pharmaceutical compositions and products) of the present invention, an amount between 0.1 to 1000 mg is administered (as a single dose or on a multi-dose, as-needed basis), dependent upon the potency of the NIF used.

Preferred embodiments encompass pharmaceutical compositions prepared for storage and subsequent administration which comprise a therapeutically effective amount of NIF or an enriched composition of NIF, as described herein in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985).

Preservatives, stabilisers, dyes and even flavouring agents may be provided in the pharmaceutical composition. For example, sodium benzoate, sorbic acid and esters of p-hydroxybenzoic acid may be added as preservatives. In addition, antioxidants and suspending agents may be used.

In employing NIFs or their pharmaceutical compositions or products in a combination therapy in vivo, the compositions/products can be administered to the mammal in a variety of ways, including parenterally (e.g. intravenously, subcutaneously, intramuscularly, colonically, rectally, nasally, buccal, transdermally, vaginally or intraperitoneally), employing a variety of dosage forms.

As will be readily apparent to one skilled in the art, the useful in vivo dosage to be administered and the particular mode of administration will vary depending upon the mammalian species treated, the particular composition employed, and the specific use for which these compositions are employed. The determination of effective dosage levels, that is the dosage levels necessary to achieve the desired result, will be within the ambit of one skilled in the art. Typically, applications of compositions are commenced at lower dosage levels, with dosage level being increased until the desired effect is achieved.

Generally, in carrying out the methods of this invention, the dosage for a NIF or its pharmaceutical compositions in combination with other active ingredient(s) can range broadly depending upon the desired effects and the therapeutic indication.

Typically, suitable dosages of NIF will be between about 0.1 and 1000 mg, preferably between about 10 and 500 mg, more preferably between about 10 and 150 mg, most preferably between about 10 and 120 mg. Administration is preferably parenteral, such as intravenous. Administration is also preferably as a single dose or on a multi-dose, as-needed basis.

Typically, suitable dosages of the combination partner compound (for example, t-PA and its variants) will be between about 0.1 and 1000 mg/kg, preferably between about 0.5 and 1.4 mg/kg, more preferably about 0.9 mg/kg. Administration is preferably parenteral, such as intravenous. Administration is also preferably as a single dose or on a multi-dose, as-needed basis.

Injectables can be prepared in conventional forms either as liquid solutions or suspensions, solid forms suitable for solution or suspension in liquid prior to injection, or as emulsions. Suitable excipients are, for example, water/saline, dextrose, mannitol, lactose, lecithin, albumin, sodium glutamate, cysteine hydrochloride or the like. In addition, if desired, the injectable pharmaceutical compositions may contain minor amounts of non-toxic auxiliary substances, such as wetting agents, pH buffering agents, and the like. If desired, absorption enhancing preparations (e.g. liposomes) may be utilised.

In carrying out the methods of the present invention, the “partner compound(s)” which will be administered in combination with at least one NIF will generally be administered to an average adult human in accordance with the generally prescribed dose, depending on the type of “partner compound(s)”, severity of the ailment and the route of administration. The “generally prescribed dose” of the “partner compound(s)” used in the methods and compositions of the present invention may be equal to, greater than or less than the dose that would be generally be administered to an average adult human when such agents are administered as single active pharmaceutical agents. Such dosages are available in the scientific and medical literature, and, for substances that have been approved for human use by the Food and Drug Administration, in the current edition (presently the 53^{rd} edition) of the Physician's Desk Reference, Medical Economics Company, Montvale, N.J., USA.

In some instances, dosage levels below the lower limit of the prescribed dose may be more than adequate, while in other cases still larger doses may be employed without causing any harmful side effects, provided that such higher dose levels are first divided into several small doses for administration throughout the dosage period (e.g. day). However, it is preferred that NIF itself be administered as a single dose (or on a multi-dose, as-needed basis).

The pharmaceutically active agents used in the methods and pharmaceutical compositions of this invention can be administered orally (which includes inhalation into the lungs), parenterally, or topically (transdermal route), alone or in combination with pharmaceutically acceptable carriers or diluents, and such administration may be carried out in single or multiple doses. More particularly, the therapeutic agents of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, injectable particulate systems, parental sustained release devices, elixirs, syrups, and the like. Such carriers include solid diluents or fillers, sterile aqueous media and various non-toxic organic solvents, etc. Moreover, oral pharmaceutical compositions can be suitably sweetened and/or flavoured. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulphate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavouring agents, colouring matter or dyes, and, if so desired, emulsifying and/or suspending agents as well, together with such diluents as water, ethanol, propylene glycol, glycerin and various like combinations thereof.

For parenteral administration, solutions of a pharmaceutically active agent used in accordance with this invention in either sesame or peanut oil or in aqueous propylene glycol may be employed. The aqueous solutions should preferably be suitably buffered (preferably between pH 4 to pH 9) if necessary and the liquid diluent first rendered isotonic. For example, NIF is used in aqueous solution at a pH of around 7. However, NIF is stable in aqueous solution down to about pH 4. The preferred “partner compound”, t-PA (or variants thereof, is generally used in aqueous solution at around pH 5. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is also possible to administer the active agents used in accordance with the present invention topically, and this may be done by way of creams, jellies, gels, pastes, patches, ointments and the like, in accordance with standard pharmaceutical practice.

It should be noted that the pharmaceutical compositions and products of the present invention may be lyophilised for storage, prior to reconstitution and thereafter administration using methods well known to those skilled in the art. Whether stored as lyophile(s) or otherwise, the active components of the combinations of the present invention may be mixed together before lyophilisation or after reconstitution (for later co-administration) or stored individually (for later simultaneous, separate or sequential administration).

Therapeutic Time Window

From the onset of the pathophysiological condition involving neutrophils, there may be a preferred or required therapeutic time window within which to administer the NIF combination therapy (including uses, methods, pharmaceutical compositions and products in accordance with the present invention) for optimal effect.

With respect to the treatment of pathophysiological conditions involving neutrophils, including stroke, the uses and methods of the present invention include the administration or co-administration of dose(s) or subsequent dose(s) within the therapeutic time window currently accepted for administration of t-PA, i.e. within about 3 h of onset of stroke (infarct). However, the NIF combination therapy (including uses, methods, pharmaceutical compositions and products) of the present invention provides for an advantageous increase in the therapeutic window. Thus, the uses and methods of the present invention include the administration or co-administration of dose(s) or subsequent dose(s), which is preferably carried out over a period of a few hours (0 to ≧ about 3 h) from onset of stroke. In a specific embodiment of the invention, the administration or co-administration of dose(s) or subsequent dose(s) is carried out over a period of 0 to > about 3 h, preferably 0 to >3 h, from onset of stroke. In another specific embodiment of the invention, the administration or co-administration of dose(s) or subsequent dose(s) is carried out over a period of 0 to ≧ about 4 h from onset of stroke. It is contemplated that administration or co-administration of dose(s) or subsequent dose(s) may be carried out over a period of 0 to ≦ about 6 h, preferably approximately 4 h to 6 h from onset of stroke. However, >6 h therapeutic time windows are also contemplated in the present invention, for example up to about 8 h, 10 h or 12 h from onset of stroke. Furthermore, the first dose(s) or subsequent dose(s) is/are preferably co-administered one or more times daily over the predetermined period.

### Advantages of the Present Invention

The present invention may have the following advantages:

The combination of at least one Neutrophil Inhibitory Factor (NIF), preferably UK-279,276, and at least one other neuroprotective or thrombolytic/fibrinolytic agent or a pharmaceutically acceptable salt thereof according to the present invention may be synergistic.

The NIFs (in particular UK-279,276) of the present invention may act as synergists.

The combination of at least one Neutrophil Inhibitory Factor (NIF), preferably UK-279,276, and at least one other neuroprotective or thrombolytic/fibrinolytic agent or a pharmaceutically acceptable salt thereof according to the present invention may increase the therapeutic time window of administration of said at least one other neuroprotective or thrombolytic/fibrinolytic agent or a pharmaceutically acceptable salt thereof.

The NIFs (in particular UK-279,276) of the present invention, in combination with at least one other neuroprotective or thrombolytic/fibrinolytic agent or a pharmaceutically acceptable salt thereof in accordance with the present invention, may afford better neuroprotection (e.g. greater reduction in infarct size and/or beneficial effect in clinical outcome) after onset of a pathophysiological condition involving neutrophils (e.g an acute cerebral infarct).

The NIFs (in particular UK-279,276) of the present invention, in combination with at least one other neuroprotective or thrombolytic/fibrinolytic agent or a pharmaceutically acceptable salt thereof in accordance with the present invention, may afford better neuroprotection (e.g. greater reduction in infarct size and/or beneficial effect in clinical outcome) after an acute cerebral infarct.

The NIFs (in particular UK-279,276) of the present invention may counteract the excitotoxic damage of a t-PA or variant thereof according to the present invention when given late (> about 3 h after onset of stroke), thus affording better neuroprotection (e.g. greater reduction in infarct size and/or beneficial effect in clinical outcome) after an acute cerebral infarct.

Definitions:

Synergy—effect of combination of compounds greater than the sum of their individual effects.

Synergist—a compound which increases the action of another.

### EXAMPLES

### Example 1

Introduction

A reduction in neuronal damage following acute ischaemic stroke can be achieved by two major strategies: restoration of cerebral blood flow through the use of thrombolytics/fibrinolytics, and inhibition of the pathophysiological cascade that occurs as a result of decreased blood flow through the use of neuroprotective agents. Therefore, combination therapy with thrombolytic/fibrinolytic and neuroprotective agents may provide additional benefit to those that can be achieved using the individual agent alone. Indeed, such benefits have already been demonstrated in several animal studies. For example, Chopp et al. recently published that the treatment combination of an anti-CD18 antibody in combination with recombinant human (rh) tissue plasminogen activator (t-PA) (rht-PA) resulted in an increased therapeutic efficacy compared to either treatment alone in a rat thromboembolic stroke model (Neurology 1999;52:273-279).

Previous studies have demonstrated that Neutrophil Inhibitory Factor (NIF) administration in a rat middle cerebral artery occlusion model of transient ischaemia produced a dose-dependent reduction in total infarct volume and improved scores measuring neurological deficit (Jiang et al. Ann Neurol 1995;38:935-42; Jiang et al. Brain Res 1998; 788:25-34). The aim of the current studies were therefore to evaluate the effects of NIF administration in a focal embolic stroke model in the rat and to determine whether the combination of NIF and rht-PA will result in a synergistic effect when both are administered 2 h after middle cerebral artery (MCA) occlusion. In addition, it was evaluated whether NIF administration can extend the therapeutic time window for rht-PA.

Methods

General procedure: Male Wistar rats (n=88) weighing 320-400 g were employed in the present study. Rats were anaesthetised with 3.5% halothane and maintained with 1.0-2.0% halothane in 70% N₂O and 30% O₂ using a face mask. Rectal temperature was maintained at 37° C. throughout the surgical procedure using a feedback-regulated water heating system. The right femoral vein was cannulated for drug administration.

Preparation of the embolus: Femoral arterial blood from a donor rat was withdrawn into 20 cm of PE-50 tubing and retained in the tube for 2 h to clot at room temperature, and subsequently retained for 22 h at 4° C. Four cm of the PE-50 tube containing clot was cut and attached at each end to a 40 mm PE 10 tube interconnected by a syringe filled with saline. The clot was shifted by continuous alternating movement from one syringe to the other for 5 minutes. A single clot (˜1 μl) was transferred to a modified PE-50 catheter with a 0.3 mm outer diameter filled with saline.

Animal model: The MCA was occluded by placement of an embolus at the origin of the MCA. Briefly, under the operating microscope (Carl Zeiss, Inc., Thornwood, N.Y., USA) the right common carotid arteries (CCA), the right external carotid artery (ECA) and the internal carotid artery (ICA) were isolated via a midline incision. A modified PE-50 catheter with a 0.3 mm outer diameter filled with a single clot, which was attached to a 100-μl Hamilton syringe filled with 0.9% saline, was introduced into the ECA lumen through a small puncture. A 15-16 mm length of catheter was gently advanced from the ECA into the lumen of the ICA. The clot in the catheter was injected into the ICA along with 2-3 μl of 0.9% saline. The catheter was withdrawn from the right ECA 5 min after injection. The right ECA was ligated.

Experimental protocols: NIF was intravenously injected at a bolus dose of 3.2 mg/kg, following by infusion at a dose of 0.2 mg/kg for 7 days. Recombinant human t-PA (rht-PA—Reteplase; Genentech, San Francisco, Calif., USA) was infused intravenously at a dose of 10 mg/kg as a 10% bolus, and the remainder was infused continuously over a 30 min interval using a Harvard pump (Harvard Apparatus, South Natick, Mass., USA). After embolization, animals were randomly divided into the following 8 groups: to examine the effect of NIF alone on ischaemia, NIF and rht-PA vehicle was administered to ischaemic rats at 2 hours (n=11) or 4 hours (n=11) after MCA occlusion; to examine the effect of rht-PA alone on ischaemia, rht-PA and saline was administered to ischaemic rats at 2 hours (n=11) or 4 hours (n=11) after MCA occlusion; to examine the effect of combination therapy of NIF and rht-PA on ischaemia, NIF and rht-PA were administered at 2 hours (n=11) or 4 hours (n=11) after MCA occlusion. Control groups consisted of ischaemic rats administered with same volume of saline and rht-PA vehicle at 2 hours (n=11) or 4 hours (n=11) after MCA occlusion.

Neurological Severity Scores (NSS): NSS is a composite of motor, sensory, reflex and balance tests. Rats were examined with NSS at 1 hours and 7 days after MCA occlusion. Neurological function was graded on a scale of 0 to 18 (normal score, 0; maximal deficit score, 18). In the severity scores of injury, 1 score point is awarded for the inability to perform the test or for the lack of a tested reflex; thus, the higher score, the more severe is the injury.

Foot-Fault test: Rats were tested for placement dysfunction of forelimbs using the modified foot-fault test (Hernandez T. D. and Schallert T., Seizures and recovery from experimental brain damage, Exp. Neurol.1988; 102:318-324) at 1 hour and 7 days after MCA occlusion. Rats were placed on elevated hexagonal grids of different sizes. Rats place their paws on the wire while moving along the grid. With each weigh-bearing step, the paw may fall or slip between the wire. This is recorded as a foot fault. The total number of steps (movement of each forelimb) that the rat used to cross the grid was counted, and the total number of foot fault for each forelimb was recorded.

Body Weight Loss: Animals were weighed before and 168 hours after embolic ischaemia.

Histopathologic studies: All the animals were anaesthetised with ketamine (44 mg/kg, intramuscularly (i.m.)) and xylazine (13 mg/kg, i.m.) and sacrificed at 7 days after MCA occlusion. Each rat was transcardially perfused with heparinized saline followed by 10% formalin. The brain was removed from the skull and cut into 7 coronal blocks, each with 2 mm thickness. The brain tissue was processed, embedded, and 6 μm thick paraffin sections from each block were cut and stained with hematoxylin and eosin (H&E) for evaluation of ischaemia cell damage. Lesion volume was measured using a Global Lab Image analysis program (Data Translation, Marlboro, Mass., USA). The area of the both hemispheres and the area containing the ischaemic neuronal damage (mm²) were calculated by tracing the area on the computer screen. The lesion volume (mm³) was determined by multiplying the appropriate area by the section interval thickness. To reduce errors associated with processing of tissue for histological analysis, the ischaemic volume is presented as the percentage of infarct volume of the contralateral hemisphere (indirect volume calculation).

Statistics: Data were analysed using a student's t-test. All values are presented as means±standard deviation (std). Statistically significance was set at p<0.05.

TABLE 1
Table explaining abbreviations used in FIGS. 4, 5 and 6.

| Abbreviation for | |
|---|---|
| treatment group | |
| NV+tV 2 | NIF vehicle 2.0 hrs + rht-PA vehicle 2.0 hrs |
| NV+tV 4 | NIF vehicle 2.0 hrs + rht-PA vehicle 4.0 hrs |
| NIF+tV 2 | NIF 2.0 hrs + rht-PA vehicle 2.0 rs |
| NIF+tV 4 | NIF 2.0 hrs + rht-PA vehicle 4.0 rs |
| NV+tPA 2 | NIF vehicle 2.0 hrs + rht-PA 2.0 hrs |
| NV+tPA 4 | NIF vehicle 2.0 hrs + rht-PA 4.0 hrs |
| NIF+tPA 2 | NIF 2.0 hrs + rht-PA 2.0 hrs |
| NIF+tPA 4 | NIF 2.0 hrs + rht-PA 4.0 hrs |

Results

Infarct Volume

Treatment with NIF had no effect on infarct volume (36.0±14.2% at 2 h and 36.1±12.6% at 4 h) compared with controls (FIG. 4). Treatment with rht-PA alone at 2 h but not 4 h significantly (P<0.05) reduced infarct volume (20.8±9.6%) (FIG. 4). Combination treatment with NIF and rht-PA at 2 h or 4 h significantly (P<0.05) reduced the infarct volume (17.4±8.1% at 2 h and 24.4±9.9% at 4 h) compared with the infarct volume (35.3±9.7% at 2 h and 38.6±9.0% at 4 h) in the control groups and the infarct volume (40.4±5.5%) in the rht-PA alone 4 h group (FIG. 4).

Neurological Functioning

A number of tests were undertaken to evaluate the neurological function of the animals after this experimental procedure. Treatment with rht-PA alone at 2 h and combination treatment of NIF and rht-PA at 2 h or 4 h produced a significant improvement in function (NSS scores and foot-fault test) when compared to the relevant control groups on day 7 (FIGS. 5 and 6). No significant difference in function across all treatment groups was observed at 1 h after MCA occlusion.

Body Weight

The experimental procedure per se resulted in weight loss in all treatment groups. Treatment with rht-PA alone at 2 h resulted in a reduced loss in body weight compared to the control group, as did the combination treatment of NIF and rht-PA at 2 h or 4 h.

Discussion

The current study examined the efficacy of NIF alone and in combination with the thrombolytic, rht-PA in a rat model of focal embolic stroke. Administration of NIF 2 h after embolization did not significantly reduce infarct volume or neurological function. The lack of efficacy of NIF in this model may reflect the fact that spontaneous thrombolysis of the embolus to give secondary reperfusion occurs late in this model (≦24 hrs) and that the ischaemic cell damage caused by the MCA occlusion is probably irreversible at that time. Thus, any further damage by reperfusion injury would be negligible and hence no benefit to NIF treatment. Such data would agree with previous findings in a model of permanent focal ischaemia, where NIF did not reduce infarct volume when compared to vehicle treated animals (Jiang et al., Brain Res 1998; 788:25-34).

However, co-administration of rht-PA and NIF after embolization produced a significant reduction in infarct volume and improved neurological functioning compared to the relevant control group. The effect on infarct volume was similar to that of administration of rht-PA alone but there was a greater improvement with respect to neurological function (NSS and foot-fault tests) of animals and the reduction in body weight was less in the combination of NIF and rht-PA given at 2 h. These data suggest that administration of NIF results in an additional improvement in functional recovery compared to the effects of rht-PA alone in this model.

Administration of rht-PA at 2 h but not 4 h was effective with respect to effects on both infarct volume and neurological functioning. These data are consistent with the efficacy of rht-PA in stroke patients where the compound is effective only when administered within 3 hours after stroke onset. However, despite the lack of efficacy of either NIF (reasons for lack of efficacy given above) or rht-PA administered alone at 2 h or 4 h respectively after embolization, the co-administration of these agents resulted in a significant reduction in infarct volume, improved neurological functioning and reduced weight loss compared to the relevant control group. These data, therefore, suggest that early administration of NIF extends the window of thrombolytic therapy for the treatment of acute stroke. These data may be clinically relevant since most stroke patients reach hospital after the current therapeutic window for rht-PA.

In summary these studies suggest that co-administration of NIF with rht-PA at 2 hours results in improved neurological functioning compared to rht-PA treatment alone. In addition, the administration of NIF at 2 hours and rht-PA at 4 hours extends the window of thrombolytic therapy for the treatment of acute stroke.

### Example 2

Formulation of NIF+t-PA Combination Product

| | Weight of Component | Weight of Component |
|---|---|---|
| Component | (mg/vial) - Liquid | (mg/vial) - Lyophile |
| UK-279,276 (NIF) | 100.00 | 100.00 |
| Human recombinant t-PA | 100.00 | 100.00 |
| Sodium dihydrogen | 4.62 | 4.62 |
| orthophosphate dihydrate | | |
| Sodium chloride | 32.8 | — |
| Disodium hydrogen | 1.47 | 1.47 |
| orthophosphate | | |
| Trehalose | — | 360 |
| Water for injections | to 4.00 ml | to 4.00 ml |
| (Ph. Eur.) | | |

Methods

1. For co-administration:

Mix UK-279,276 (NIF)+human recombinant t-PA with water (for injections) and excipients (as listed above for Liquid or Lvophile, as appropriate) to required volume.

For sterile injectable solution (Liquid):

Filter the resulting solution through a sterile 0.22 μm nylon filter into sterile glass vial(s) and seal aseptically.

Storage conditions: 2-8° C.

Administer parentally, e.g. intravenously, subcutaneously or intramuscularly.

For Lyophile:

Filter the resulting solution through a sterile 0.22 μm nylon filter into a sterile container.

Fill 4 ml volumes into sterile freeze-drying vials and stopper.

Lyophilise.

Storage conditions: 2-8° C.

Reconstitute with water (for injections) to produce a sterile injectable solution.

Administer parentally, e.g. intravenously, subcutaneously or intramuscularly.

2. For individual (simultaneous, separate or sequential) administration:

Mix UK-279,276 (NIF) with water (for injections) and excipients (as listed above for Liquid or Lyophile, as appropriate) to required volume.

Mix human recombinant t-PA with water (for injections) and excipients (as listed above for Liquid or Lyophile, as appropriate) to required volume.

For sterile injectable solution (Liquid):

Filter the resulting solutions through a sterile 0.22 μm nylon filter into separate sterile glass vials and seal aseptically.

Storage conditions: 2-8° C.

Administer parentally, e.g. intravenously, subcutaneously or intramuscularly.

For Lyophile:

Filter the resulting solutions through a sterile 0.22 μm nylon filter into separate sterile containers.

Fill 4 ml volumes into sterile freeze-drying vials and stopper.

Lyophilise.

Storage conditions: 2-8° C.

Reconstitute with water (for injections) to produce a sterile injectable solution.

Administer parentally, e.g. intravenously, subcutaneously or intramuscularly.

It will be appreciated that the foregoing is provided by way of example only and modification of detail may be made without departing from the scope of the invention.

For the avoidance of doubt, all references disclosed herein are incorporated by reference.

1. Use of a combination of at least one Neutrophil Inhibitory Factor (NIF) and at least one other neuroprotective or thrombolytic/fibrinolytic agent or a pharmaceutically acceptable salt thereof in the manufacture of a medicament for the treatment of pathophysiological conditions involving neutrophils.

2. Use according to claim 1, wherein said Neutrophil Inhibitory Factor (NIF) has the amino acid sequence as set out in SEQ ID NO: 3 or 4 or a fragment, variant, homologue, derivative or analogue thereof.

3. Use according to claim 1 or claim 2, wherein said Neutrophil Inhibitory Factor (NIF) is UK-279,276.

4. Use according to any one of claims 1 to 3, wherein said pathophysiological condition involving neutrophils is ischaemic damage and/or reperfusion injury.

5. Use according to claim 4, wherein said ischaemic damage and/or reperfusion injury is stroke, traumatic head injury, post-ischaemic-reperfusion injury, post-ischaemic cerebral inflammation or ischaemia-reperfusion injury following myocardial infarction.

6. Use according to any one of claims 1 to 5, wherein said neuroprotective or thrombolytic/fibrinolytic agent(s) is/are any one or more of a plasminogen activator, urokinase, pro-urokinase, streptokinase, p-anisoylated plasminogen streptokinase activator complex (APSAC), urokinase plasminogen activator (uPA), a MMP inhibitor, a sodium channel antagonist, a nitric oxide synthase (NOS) inhibitor, a NMDA receptor antagonist, a NMDA glycine site receptor antagonist, a potassium channel opener, an AMPA/kainate receptor antagonist, a calcium channel antagonist, a GABA_{A} receptor modulator, a GABA_{A} receptor agonist, an SSRI, a 5-HT_{1A} agonist or an anti-inflammatory agent.

7. Use according to claim 6, wherein said plasminogen activator is tissue plasminogen activator (t-PA) or variants thereof or Desmoteplase.

8. Use according to claim 7, wherein said variants of tissue plasminogen activator (t-PA) are Alteplase, Monteplase, Reteplase, Lanoteplase, Duteplase and Tenecteplase.

9. Use according to claim 8, wherein said variant of tissue plasminogen activator (t-PA) is Alteplase, Monteplase or Tenecteplase and said pathophysiological condition involving neutrophils is stroke.

10. Use according to any one of claims 1 to 9, wherein said pathophysiological condition involving neutrophils is stroke and the therapeutic time window of administration of said at least one other neuroprotective or thrombolytic/fibrinolytic agent is 0 to >about 3 h from onset of stroke.

11. A method of treating pathophysiological conditions involving neutrophils, comprising administering to a subject in need of said treatment, either simultaneously, separately or sequentially, a combination of:
(a) at least one Neutrophil Inhibitory Factor (NIF); and
(b) at least one other neuroprotective or thrombolytic/fibrinolytic agent or a pharmaceutically acceptable salt thereof;
wherein the two or more agents of (a) or (b) above are present in amounts that render the combination of said two or more agents effective in treating pathophysiological conditions involving neutrophils.

12. The method according to claim 11, wherein said Neutrophil Inhibitory Factor (NIF) has the amino acid sequence as set out in SEQ ID NO: 3 or 4 or a fragment, variant, homologue, derivative or analogue thereof.

13. The method according to claim 11 or claim 12, wherein said Neutrophil Inhibitory Factor (NIF) is UK-279,276.

14. The method according to any one of claims 11 to 13, wherein said pathophysiological condition involving neutrophils is ischaemic damage and/or reperfusion injury.

15. The method according to claim 14, wherein said ischaemic damage and/or reperfusion injury is stroke, traumatic head injury, post-ischaemic-reperfusion injury, post-ischaemic cerebral inflammation or ischaemia-reperfusion injury following myocardial infarction.

16. The method according to any one of claims 11 to 15, wherein said neuroprotective or thrombolytic/fibrinolytic agent(s) is/are any one or more of a plasminogen activator, urokinase, pro-urokinase, streptokinase, p-anisoylated plasminogen streptokinase activator complex (APSAC), urokinase plasminogen activator (uPA), a MMP inhibitor, a sodium channel antagonist, a nitric oxide synthase (NOS) inhibitor, a NMDA receptor antagonist, a NMDA glycine site receptor antagonist, a potassium channel opener, an AMPA/kainate receptor antagonist, a calcium channel antagonist, a GABA_{A} receptor modulator, a GABA_{A} receptor agonist, an SSRI, a 5-HT_{1A} agonist or an anti-inflammatory agent.

17. The method according to claim 16, wherein said plasminogen activator is tissue plasminogen activator (t-PA) or variants thereof or Desmoteplase.

18. The method according to claim 17, wherein said variants of tissue plasminogen activator (t-PA) are Alteplase, Monteplase, Reteplase, Lanoteplase, Duteplase and Tenecteplase.

19. The method according to claim 18, wherein said variant of tissue plasminogen activator (t-PA) is Alteplase, Monteplase or Tenecteplase and said pathophysiological condition involving neutrophils is stroke.

20. The method according to any one of claims 11 to 19, wherein said pathophysiological condition involving neutrophils is stroke and the therapeutic time window of administration of said at least one other neuroprotective or thrombolytic/fibrinolytic agent is 0 to >about 3 h from onset of stroke.

21. A pharmaceutical composition comprising:
(a) at least one Neutrophil Inhibitory Factor (NIF);
(b) at least one other neuroprotective or thrombolytic/fibrinolytic agent or a pharmaceutically acceptable salt thereof; and optionally
(c) a pharmaceutically acceptable carrier, diluent, excipient or adjuvant.

22. The pharmaceutical composition according to claim 21, wherein said Neutrophil Inhibitory Factor (NIF) has the amino acid sequence as set out in SEQ ID NO: 3 or 4 or a fragment, variant, homologue, derivative or analogue thereof.

23. The pharmaceutical composition according to claim 21 or claim 22, wherein said Neutrophil Inhibitory Factor (NIF) is UK-279,276.

24. The pharmaceutical composition according to any one of claims 21 to 23 for use in the treatment of pathophysiological conditions involving neutrophils.

25. The pharmaceutical composition according to claim 24, wherein said pathophysiological condition involving neutrophils is ischaemic damage and/or reperfusion injury.

26. The pharmaceutical composition according to claim 25, wherein said ischaemic damage and/or reperfusion injury is stroke, traumatic head injury, post-ischaemic-reperfusion injury, post-ischaemic cerebral inflammation or ischaemia-reperfusion injury following myocardial infarction.

27. The pharmaceutical composition according to any one of claims 21 to 26, wherein the neuroprotective or thrombolytic/fibrinolytic agent(s) is/are any one or more of a plasminogen activator, urokinase, pro-urokinase, streptokinase, p-anisoylated plasminogen streptokinase activator complex (APSAC), urokinase plasminogen activator (uPA), a MMP inhibitor, a sodium channel antagonist, a nitric oxide synthase (NOS) inhibitor, a NMDA receptor antagonist, a NMDA glycine site receptor antagonist, a potassium channel opener, an AMPA/kainate receptor antagonist, a calcium channel antagonist, a GABA_{A} receptor modulator, a GABA_{A} receptor agonist, an SSRI, a 5-HT_{1A} agonist or an anti-inflammatory agent.

28. The pharmaceutical composition according to claim 27, wherein said plasminogen activator is tissue plasminogen activator (t-PA) or variants thereof or Desmoteplase.

29. The pharmaceutical composition according to claim 28, wherein said variants of tissue plasminogen activator (t-PA) are Alteplase, Monteplase, Reteplase, Lanoteplase, Duteplase and Tenecteplase.

30. The pharmaceutical composition according to claim 29, wherein said variant of tissue plasminogen activator (t-PA) is Alteplase, Monteplase or Tenecteplase and said pathophysiological condition involving neutrophils is stroke.

31. The pharmaceutical composition according to any one of claims 21 to 30, wherein said pathophysiological condition involving neutrophils is stroke and the therapeutic time window of administration of said at least one other neuroprotective or thrombolytic/fibrinolytic agent is 0 to >about 3 h from onset of stroke.

32. A process for preparing the pharmaceutical composition according to any one of claims 21 to 31, comprising the steps of:
(a) performing an assay to identify one or more agents that is/are, or has/have the capability of acting as, Neutrophil Inhibitory Factor (NIF);
(b) admixing one or more of said agent(s) with one or more other neuroprotective or thrombolytic/fibrinolytic agent(s); and optionally admixing
(c) a pharmaceutically acceptable carrier, diluent, excipient or adjuvant therewith.

33. The process according to claim 32, wherein said process also includes the subsequent step of:
(d) administering said pharmaceutical composition to a subject in need of the same.

34. The process according to claim 32 or claim 33, wherein said Neutrophil Inhibitory Factor (NIF) has the amino acid sequence as set out in SEQ ID NO: 3 or 4 or a fragment, variant, homologue, derivative or analogue thereof.

35. The process according to any of claims 32 to 34, wherein said Neutrophil Inhibitory Factor (NIF) is UK-279,276.

36. The process according to any one of claims 32 to 35, wherein the neuroprotective or thrombolytic/fibrinolytic agent(s) is/are any one or more of a plasminogen activator, urokinase, pro-urokinase, streptokinase, p-anisoylated plasminogen streptokinase activator complex (APSAC), urokinase plasminogen activator (uPA), a MMP inhibitor, a sodium channel antagonist, a nitric oxide synthase (NOS) inhibitor, a NMDA receptor antagonist, a NMDA glycine site receptor antagonist, a potassium channel opener, an AMPA/kainate receptor antagonist, a calcium channel antagonist, a GABA_{A} receptor modulator, a GABA_{A} receptor agonist, an SSRI, a 5-HT_{1A} agonist or an anti-inflammatory agent.

37. The process according to claim 36, wherein said plasminogen activator is tissue plasminogen activator (t-PA) or variants thereof or Desmoteplase.

38. The process according to claim 37, wherein said variants of tissue plasminogen activator (t-PA) are Alteplase, Monteplase, Reteplase, Lanoteplase, Duteplase and Tenecteplase.

39. The process according to claim 38, wherein said variant of tissue plasminogen activator (t-PA) is Alteplase, Monteplase or Tenecteplase.

40. Products containing: p1 (a) at least one Neutrophil Inhibitory Factor (NIF); and
(b) at least one other neuroprotective or thrombolytic/fibrinolytic agent or a pharmaceutically acceptable salt thereof;
as a combined preparation for simultaneous, separate or sequential use in treating pathophysiological conditions involving neutrophils.

41. The products according to claim 40, wherein said Neutrophil Inhibitory Factor (NIF) has the amino acid sequence as set out in SEQ ID NO: 3 or 4 or a fragment, variant, homologue, derivative or analogue thereof.

42. The products according to claim 40 or claim 41, wherein said Neutrophil Inhibitory Factor (NIF) is UK-279,276.

43. The products according to any one of claims 40 to 42, wherein said pathophysiological condition involving neutrophils is ischaemic damage and/or reperfusion injury.

44. The products according to claim 43, wherein said ischaemic damage and/or reperfusion injury is stroke, traumatic head injury, post-ischaemic-reperfusion injury, post-ischaemic cerebral inflammation or ischaemia-reperfusion injury following myocardial infarction.

45. The products according to any one of claims 40 to 44, wherein the neuroprotective or thrombolytic/fibrinolytic agent(s) is/are any one or more of a plasminogen activator, urokinase, pro-urokinase, streptokinase, p-anisoylated plasminogen streptokinase activator complex (APSAC), urokinase plasminogen activator (uPA), a MMP inhibitor, a sodium channel antagonist, a nitric oxide synthase (NOS) inhibitor, a NMDA receptor antagonist, a NMDA glycine site receptor antagonist, a potassium channel opener, an AMPA/kainate receptor antagonist, a calcium channel antagonist, a GABA_{A} receptor modulator, a GABA_{A} receptor agonist, an SSRI, a 5-HT_{1A} agonist or an anti-inflammatory agent.

46. The products according to claim 45, wherein said plasminogen activator is tissue plasminogen activator (t-PA) or variants thereof or Desmoteplase.

47. The products according to claim 46, wherein said variants of tissue plasminogen activator (t-PA) are Alteplase, Monteplase, Reteplase, Lanoteplase, Duteplase and Tenecteplase.

48. The products according to claim 47, wherein said variant of tissue plasminogen activator (t-PA) is Alteplase, Monteplase or Tenecteplase and said pathophysiological condition involving neutrophils is stroke.

49. The products according to any one of claims 40 to 48, wherein said pathophysiological condition involving neutrophils is stroke and the therapeutic time window of administration of said at least one other neuroprotective or thrombolytic/fibrinolytic agent is 0 to >about 3 h from onset of stroke.


SEQUENCE LISTING (7 sequences)

SEQ ID NO: 1 - DNA, 1845 bp, Ancylostoma caninum (canine hookworm)
agttctcagatagtcacagtagcccttcttttcattgtacacaagtgaagatgggcacttcatggtagtcgcgactccttcattacagtaaacatagtcggatgtgcatcccaacgaatagtagccattctgctttgtcttgcagtcaacggtcttcgcaatttgtggtacagcagcaggagccggaggctgcatcgctggagctgctggtggagctggcacaacagaagccggaggtggagcaaccagttcaggcgtgcagttctcaggatagtcgcagtagcccttcttctcatggtatacaagtgaagaatggaggcctatcttgtggtcttaattgccattgctggcatagctcattccaatgaacacaacctgaggtgcccgcagaatggaacagaaatgcccggtttcaacgactcgattaggcttcaatttttagcaatgcacaatggttacagatcaaaacttgcgctaggtcacatcagcataactgaagaatccgaaagtgacgatgatgacgatttcggttttttacccgatttcgctccaagggcatcgaaaatgagatatctggaatatgactgtgaagctgaaaaaagcgcctacatgtcggctagaaattgctcggacagttcttctccaccagagggctacgatgaaaacaagtatattttcgaaaactcaaacaatatcagtgaagctgctctgaaggccatgatctcgtgggcaaaagaggctttcaacctaaataaaacaaaagaaggagaaggagttctgtaccggtcgaaccacgacatatcaaacttcgctaatctggcttgggacgcgcgtgaaaagtttggttgtcgcagttgttaactgccctttgggagaaatcgatgatgaaaccaaccatgatggagaaacctatgcaacaaccatccatgtagtctgccactacccgaaaataaacaaaactgaaggacagccgatttacaaggtagggacaccatgcgacgattgcagtgatacacaaaaaaagcagacaataccacgtctgcggatccggtgtgtattccggatgacggagtctgctttattggctcgaaagccgattacgatagcaaggagttttatcgattccgagagttatgaataagtcgagacgtataaagaagccaaggcaacgtaagcgagcaagtctcgaagacgatggagtcagcgaaagaggcggctgccaaagttggcgagcaggtgtcagattttttccaagggaacccattttccacgcctgtgggccgcaagatagaacttgccacgaacgcttcgattcttgcactgagaattggggtttgaacatggaaatctgtgatttcgtcaataacactgaggacggtgccaaagatgctgtacgggctattcgcaaacgtctgcacacaaatatgtgtaagaataacgcaatcgtcatgtacacattaacggtgctggagacgtgcgtgaagaactgtggccataatttccacgtgctcgtatgttccaaggactttgtgcaggatttggtgaagttgatcggctcgaagttcgatacgcctcagattattcacgagcgtgtattgtcacttattcaggcttgggcagatgcattccgcaatcaaccagatcttcagggagtcgtacaggtctatgaagaacttgttagtaagggggttacattccctgcaactgatctagacgctatggcacctatactaacaccaaaacaaacagtcttcactgagccaaaggcatcaacggctgttccttcgcagtcaggtggaggacctagttacgaggtggtcagccaaccagatggtccaatttact

SEQ ID NO: 2 - DNA, 825 bp, Ancylostoma caninum (canine hookworm)
atggaggcctatcttgtggtcttaattgccattgctggcatagctcattccaatgaacacaacctgaggtgcccgcagaatggaacagaaatgcccggtttcaacgactcgattaggcttcaatttttagcaatgcacaatggttacagatcaaaacttgcgctaggtcacatcagcataactgaagaatccgaaagtgacgatgatgacgatttcggttttttacccgatttcgctccaagggcatcgaaaatgagatatctggaatatgactgtgaagctgaaaaaagcgcctacatgtcggctagaaattgctcggacagttcttctccaccagagggctacgatgaaaacaagtatattttcgaaaactcaaacaatatcagtgaagctgctctgaaggccatgatctcgtgggcaaaagaggctttcaacctaaataaaacaaaagaaggagaaggagttctgtaccggtcgaaccacgacatatcaaacttcgctaatctggcttgggacgcgcgtgaaaagtttggttgcgcagttgttaactgccctttgggagaaatcgatgatgaaaccaaccatgatggagaaacctatgcaacaaccatccatgtagtctgccactacccgaaaataaacaaaactgaaggacagccgatttacaaggtagggacaccatgcgacgattgcagtgaatacacaaaaaaagcagacaataccacgtctgcggatccggtgtgtattccggatgacggagtctgctttattggctcgaaagccgattacgatagcaaggagttttatcgattccgagagttatga

SEQ ID NO: 3 - protein, 274 aa, Ancylostoma caninum (canine hookworm)
MEAYLVVLIAIAGIAHSNEHNLRCPQNGTEMPGFNDSIRLQFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFREL

SEQ ID NO: 4 - protein, 257 aa, Ancylostoma caninum (canine hookworm)
NEHNLRCPQNGTEMPGFNDSIRLQFLAMHNGYRSKLALGHISITEESESDDDDDFGFLPDFAPRASKMRYLEYDCEAEKSAYMSARNCSDSSSPPEGYDENKYIFENSNNISEAALKAMISWAKEAFNLNKTKEGEGVLYRSNHDISNFANLAWDAREKFGCAVVNCPLGEIDDETNHDGETYATTIHVVCHYPKINKTEGQPIYKVGTPCDDCSEYTKKADNTTSADPVCIPDDGVCFIGSKADYDSKEFYRFREL

SEQ ID NO: 5 - DNA, 2519 bp, Homo sapiens
acagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatactgtacttaataaattcagatatatttttcacagtttttcc

SEQ ID NO: 6 - DNA, 1689 bp, Homo sapiens
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtga

SEQ ID NO: 7 - protein, 562 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP